(19) United States
(12) Patent Application Publication
(10) Pub. No.: US 2025/0064990 A1
(43) Pub. Date: Feb. 27, 2025

(54) METHODS OF SCREENING FOR VMAT2 INHIBITORS

(71) Applicant: Neurocrine Biosciences, Inc., San Diego, CA (US)

(72) Inventors: Ryan Terry-Lorenzo, San Diego, CA (US); Dietrich Haubenberger, San Diego, CA (US); Nagdeep Giri, San Diego, CA (US)

(21) Appl. No.: 18/684,426

(22) PCT Filed: Aug. 18, 2022
(86) PCT No.: PCT/US2022/075137

§ 371 Date: Feb. 16, 2024

Related U.S. Application Data
(60) Provisional application No. 63/235,407, filed on Aug. 20, 2021.

(51) Int. Cl. A61K 51/04

(52) U.S. Cl. CPC A61K 51/0455; A61K 2123/00

(57) ABSTRACT

The present application is related to methods of preparing pharmaceutical compositions comprising a VMAT2 inhibitor and/or identifying therapeutically effective dosages of a VMAT2 inhibitor, wherein the VMAT2 inhibitor is useful for treating neurological and psychiatric diseases and disorders and for achieving an occupancy rate between 80-96% in a subject.

### CROSS-REFERENCE TO RELATED APPLICATIONS

This application claims the benefit of U.S. Provisional Application Ser. No. 63/235,407, filed Aug. 20, 2021, the disclosure of which is incorporated herein by reference in its entirety.

### TECHNICAL FIELD

The present application is related to methods of preparing pharmaceutical compositions comprising a VMAT2 inhibitor and/or identifying therapeutically effective dosages of a VMAT2 inhibitor, wherein the VMAT2 inhibitor is useful for treating neurological and psychiatric diseases and disorders and for achieving an occupancy rate between 80-96% in a subject.

### BACKGROUND

Dysregulation of dopaminergic systems is integral to several central nervous system (CNS) disorders, including neurological and psychiatric diseases and disorders. These neurological and psychiatric diseases and disorders include hyperkinetic movement disorders, and conditions such as schizophrenia and mood disorders. The transporter protein vesicular monoamine transporter-2 (VMAT2) plays an important role in presynaptic dopamine release and regulates monoamine uptake from the cytoplasm to the synaptic vesicle for storage and release.

### SUMMARY

The present application provides, inter alia, a method of preparing a pharmaceutical composition comprising a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, wherein a VMAT2 occupancy rate between 80-96% is indicative of the amount of the VMAT2 inhibitor being a therapeutically effective dosage; and
(c) admixing the therapeutically effective dosage of the VMAT2 inhibitor with a pharmaceutically acceptable carrier.

The present application further provides a method of identifying a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, and
(c) identifying a therapeutically effective dosage of a VMAT2 inhibitor when the VMAT2 occupancy rate of the amount of the VMAT2 inhibitor is between 80-96%.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIGS. 1A-1C show [¹⁸F]AV-133 SUV images averaged between 60-120 min at baseline and post NBI-750142 at different doses in three different cynomolgus monkeys: GC786 (FIG. 1A), EC865 (FIG. 1B) and LC206 (FIG. 1C). A dose-dependent decrease in striatal uptake was observed.

FIG. 1D shows a Cynomolgus MR image for anatomical reference in comparing the data of FIGS. 1A-1C.

FIG. 2 shows [¹⁸F]AV-133 time-activity curves comparing baseline with different dose levels of NBI-750142 from the NHP model described in Example 1.

FIG. 3A shows the relationship of target occupancy and total NBI-750142 plasma concentration in the NHP model described in Example 1.

FIG. 3B shows estimated VMAT2 target occupancy (% TO) for human doses of NBI-750142 from the NHP model described in Example 1.

FIGS. 4A-4B show [¹⁸F]AV-133 SUV images averaged between 90-120 min at baseline and post NBI-98782 at different doses in two different cynomolgus monkeys: A7701 (FIG. 2A) and A7702 (FIG. 2B). A dose-dependent decrease in striatal uptake was observed.

FIG. 5 shows [¹⁸F] AV-133 time-activity curves comparing baseline with different dose levels of NBI-98782 from the NHP model described in Example 2.

FIG. 6 shows the relationship of target occupancy and total NBI-98782 plasma concentration from the NHP model described in Example 2.

FIGS. 7A-7F show [¹⁸F]AV-133 SUVR (occipital reference region) images averaged between 60-120 min at baseline, and post NBI-750142 dose in human at 1.5 h (T1) and 18 h (T2). Cohort 1: 100 mg (FIG. 7A); Cohort 2: 200 mg (FIG. 7B); Cohort 3: 60 mg (FIG. 7C); Cohort 4: 60 mg (FIG. 7D); Cohort 5A: 10 mg (FIG. 7E); and Cohort 5B: 10 mg (FIG. 7F). The MRI images are shown for anatomical reference.

FIGS. 8A-8F show [¹⁸F]AV-133 SUV time-activity curves at baseline, and post NBI-750142 dose in human at 1.5 h (T1) and 18 h (T2). Cohort 1: 100 mg (FIG. 8A); Cohort 2: 200 mg (FIG. 8B); Cohort 3: 60 mg (FIG. 8C); Cohort 4: 60 mg (FIG. 8D); Cohort 5A: 10 mg (FIG. 8E); and Cohort 5B: 10 mg (FIG. 8F).

FIGS. 9A-9B show the relationship of NBI-750142 striatal occupancy vs total plasma concentration based on the Eₘₐₓ model for T1 and T2 together (FIG. 9A), T1 only (FIG. 9B).

FIGS. 10A-10B show a comparison of the Eₘₐₓ and Upregulation model fits to striatal occupancy at T1 and T2 (FIG. 10A) and T1 only vs total plasma concentration (FIG. 10B) across cohorts and subjects.

FIG. 11 shows estimated VMAT2 target occupancy (% TO) for NBI-98782 from the NHP model described in Example 2.

FIG. 12. shows estimated VMAT2 occupancy of valbenazine throughout each 24-hour period. FIG. 12 also shows cynomolgus monkey pharmacokinetic data with estimations of % TO at select timepoints. As described below, there were adverse events at these doses, enabling a benchmarking of % TO values that could lead to adverse events.

FIG. 13A shows estimated VMAT2 concentration-dependent target occupancy (% TO) for NBI-98782 (EC₅₀ 1.5 ng/mL). FIG. 13B shows estimated VMAT2 concentration-dependent target occupancy (% TO) for NBI-98782 (EC₅₀ 3.8 ng/mL).

FIG. 14 shows nonhuman primate imaging and sampling procedures.

FIG. 15 shows a protocol for estimating VMAT2 occupancy.

FIG. 16 shows concentration vs time curves for [+]-α-HTBZ during PET scan sessions.

FIG. 17 shows percent target occupancy achieved in a nonhuman primate study.

### DETAILED DESCRIPTION

The present application is related to methods of preparing pharmaceutical compositions comprising a VMAT2 inhibitor and/or identifying therapeutically effective dosages of a VMAT2 inhibitor. In some embodiments, the VMAT2 inhibitor provided herein is particularly useful for achieving an occupancy rate between 80-96% in a subject and for treating neurological and psychiatric diseases and disorders as described herein. There is a significant, unmet need for identifying therapeutically effective dosages of VMAT2 inhibitors that afford an occupancy rate between 80-96% (e.g., to reduce or prevent treatment emergent adverse events (TEAEs) associated with VMAT2 inhibition). The present disclosure fulfills these and other needs, as evident in reference to the following disclosure.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.”

Headings provided herein are for convenience only and do not interpret the scope or meaning of the claimed invention. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

### Methods of Use

Accordingly, the present application provides a method of preparing a pharmaceutical composition comprising a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, wherein a VMAT2 occupancy rate between 80-96% is indicative of the amount of the VMAT2 inhibitor being a therapeutically effective dosage; and
(c) admixing the therapeutically effective dosage of the VMAT2 inhibitor with a pharmaceutically acceptable carrier.

The present application further provides a method of identifying a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, and
(c) identifying a therapeutically effective dosage of a VMAT2 inhibitor when the VMAT2 occupancy rate of the amount of the VMAT2 inhibitor is between 80-96%.

In some embodiments, VMAT2 occupancy is measured by one or more imaging techniques. In some embodiments, the one or more imaging techniques comprise administering to the subject an imaging agent capable of binding VMAT2 and subsequently imaging the subject.

In some embodiments, the imaging agent is a VMAT2 inhibitor.

In some embodiments, VMAT2 occupancy is measured by a positron emission tomography (PET) assay. In some embodiments, the PET assay comprises administering to the subject a PET imaging agent capable of binding VMAT2 and subsequently imaging the subject.

In some embodiments, the PET assay comprises:
(a) administering to the subject a PET imaging agent capable of binding VMAT2;
(b) waiting a time sufficient for the PET imaging agent to bind VMAT2;
(c) imaging the subject one or more times;
(d) measuring the VMAT2 displacement of the PET imaging agent; and
(e) determining VMAT2 occupancy based on the measured VMAT2 displacement of the PET imaging agent.

In some embodiments, the VMAT2 displacement of the PET imaging agent is measured at one or more time points during the imaging.

In some embodiments, the PET assay further comprises imaging the subject prior to step (a) to obtain a baseline image. In some embodiments, the VMAT2 inhibitor is administered to the subject after step (a). In some embodiments, the VMAT2 inhibitor is administered to the subject after step (b). In some embodiments, the VMAT2 inhibitor is administered to the subject after step (b) and prior to step (c).

In some embodiments, the PET imaging agent is a radiolabeled VMAT2 inhibitor. In some embodiments, the PET imaging agent is a [¹¹C]-radiolabeled VMAT2 inhibitor. In some embodiments, the PET imaging agent is a [¹⁸F]-radiolabeled VMAT2 inhibitor.

In some embodiments, the PET imaging agent is a radiolabeled analog of a VMAT2 inhibitor selected from the group consisting of valbenazine, tetrabenazine, deutetrabenazine, dihydrotetrabenazine, NBI-750142, and AV-133.

In some embodiments, the PET imaging agent is a [¹¹C]- or [¹⁸F]-radiolabeled analog of a VMAT2 inhibitor selected from the group consisting of valbenazine, tetrabenazine, deutetrabenazine, dihydrotetrabenazine, NBI-750142, and AV-133. In some embodiments, the radiolabeled analog of dihydrotetrabenazine is a radiolabeled analog of (+)-α-dihydrotetrabenazine.

In some embodiments, the PET imaging agent is [¹⁸F]-AV-133.

In some embodiments, the methods provided herein further comprise measuring the plasma concentration of the VMAT2 inhibitor in the subject. In some embodiments, the plasma concentration of the VMAT2 inhibitor is measured at one or more time points during the imaging of step (c). In some embodiments, the plasma concentration of the VMAT2 inhibitor is measured at one or more time points prior to the imaging of step (c). In some embodiments, the plasma concentration of the VMAT2 inhibitor is measured at one or more time points during the imaging of step (c) and prior to the imaging of step (c).

In some embodiments, the plasma concentration of the VMAT2 inhibitor is measured at one or more time points from about two hours prior to the imaging of step (c) until the end of the imaging.

In some embodiments, the plasma concentration of the VMAT2 inhibitor is measured at one or more time points from about one hour prior to the imaging of step (c) until the end of the imaging.

In some embodiments, the dose administered is identified as a therapeutically effective dosage if the VMAT2 occupancy is determined to be from at least 80% to about 96%, for example, about 80%, about 82%, about 84%, about 86%, about 88%, about 90%, about 92%, about 94%, or about 96%.

In some embodiments, the dose administered is identified as a therapeutically effective dosage if the VMAT2 occupancy is determined to be from at least 80% and no more than 96%.

In some embodiments, the dose administered is identified as a therapeutically effective dosage if the VMAT2 occupancy is determined to be from at least 80% and no more than 94%.

In some embodiments, the dose administered is identified as a therapeutically effective dosage if the VMAT2 occupancy is determined to be from at least 80% and no more than 92%.

In some embodiments, the dose administered is identified as a therapeutically effective dosage if the VMAT2 occupancy is determined to be from at least 80% and no more than 90%.

In some embodiments, the method provided herein further comprises monitoring the subject for one or more symptoms associated with a treatment-emergent adverse event (TEAE) after administration of the VMAT2 inhibitor.

In some embodiments, the monitoring is performed for about 30 minutes to about 24 hours after administration of the VMAT2 inhibitor, for example, about 30 minutes, about 60 minutes, about 90 minutes, about 120 minutes, about 180 minutes, about 6 hours, about 12 hours, about 18 hours, or about 24 hours.

In some embodiments, the monitoring is performed for about 30 minutes to about 90 minutes after administration of the VMAT2 inhibitor.

In some embodiments, the monitoring is performed for about 30 minutes to about 60 minutes after administration of the VMAT2 inhibitor.

In some embodiments, the method provided herein further comprises identifying the subject as not exhibiting one or more symptoms associated with a TEAE after administration of the VMAT2 inhibitor.

In some embodiments, the method provided herein further comprises identifying the subject as not exhibiting one or more symptoms selected from ptosis, decreased activity, sedation, anxiety, nausea, akathisia, and salivation after administration of the VMAT2 inhibitor.

In some embodiments, the method provided herein further comprises identifying the subject as not exhibiting one or more symptoms selected from ptosis, decreased activity, and salivation after administration of the VMAT2 inhibitor.

In some embodiments, the subject has been identified as not exhibiting one or more symptoms associated with a TEAE after administration of the VMAT2 inhibitor.

In some embodiments, the subject has been identified as not exhibiting one or more symptoms selected from ptosis, decreased activity, sedation, anxiety, nausea, akathisia, and salivation after administration of the VMAT2 inhibitor.

In some embodiments, the subject has been identified as not exhibiting one or more symptoms selected from ptosis, decreased activity, and salivation after administration of the VMAT2 inhibitor.

In some embodiments, the dose administered is identified as a therapeutically effective dosage if VMAT2 occupancy is determined to be from at least 80% and no more than 96%, for example, about 80%, about 82%, about 84%, about 86%, about 88%, about 90%, about 92%, about 94%, or about 96%.

In some embodiments, the dose administered is identified as a therapeutically effective dosage if VMAT2 occupancy is determined to be from at least 85% and no more than 95%.

In some embodiments, the dose administered is identified as a therapeutically effective dosage if VMAT2 occupancy is determined to be from at least 85% and no more than 90%.

In some embodiments, the methods described above further comprise optionally measuring synaptic dopamine in the subject by
(i) administering radioligand [¹¹C](+)4-propyl-3,4,4a,5,6,10b-hexahydro-2H-naphtho[1,2-b][1,4]oxazin-9-ol ([¹¹C]-PHNO) to the subject; and
(ii) image scanning the subject;
wherein a 20-45% increase in ([¹¹C]-PHNO binding potential relative to the non-displaceable binding ([¹¹C]-PHNO BP_{ND}) corresponds to a decrease in synaptic dopamine and is indicative of the amount of the VMAT2 inhibitor being a therapeutically effective dosage.

The present application further provides a method of preparing a pharmaceutical composition comprising a therapeutically effective dosage of a VMAT2 inhibitor, the method comprising:
admixing the therapeutically effective dosage of the VMAT2 inhibitor with a pharmaceutically acceptable carrier;
wherein the therapeutically effective dosage of the VMAT2 inhibitor was identified by
measuring in vivo VMAT2 occupancy of the VMAT2 inhibitor in a subject previously administered with an amount of the VMAT2 inhibitor, wherein a VMAT2 occupancy rate between 80-96% was indicative of the amount of the VMAT2 inhibitor being a therapeutically effective dosage.

In some embodiments, the method further comprises optionally measuring synaptic dopamine in the subject by
(i) administering radioligand [¹¹C](+)4-propyl-3,4,4a,5,6,10b-hexahydro-2H-naphtho[1,2-b][1,4]oxazin-9-ol ([¹¹C]-PHNO) to the subject; and
(ii) image scanning the subject;
wherein a 20-45% increase in ([¹¹C]-PHNO binding potential relative to the non-displaceable binding ([¹¹C]-PHNO BP_{ND}) corresponded to a decrease in synaptic dopamine and was indicative of the amount of the VMAT2 inhibitor being a therapeutically effective dosage.

In some embodiments, the VMAT2 inhibitor dose administered is identified as a therapeutically effective dosage if the increase in [¹¹C]-PNHO BP_{ND} is determined to be from at least 10% to about 60%, for example, about 10%, about 15%, about 20%, about 25%, about 30%, about 35%, about 40%, about 45%, about 50%, about 55% or about 60%. In such embodiments, the increase in [¹¹C]-PNHO BP_{ND} corresponds to a decrease in synaptic dopamine.

The term “compound” as used herein (e.g., a VMAT2 inhibitor) is meant to include all stereoisomers, geometric isomers, tautomers, and isotopes of the structures depicted. Compounds herein identified by name or structure as one particular tautomeric form are intended to include other tautomeric forms unless otherwise specified.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate.

A substance is an “inhibitor” of enzyme activity when the specific activity or the metabolic effect of the specific activity of the enzyme can be decreased by the presence of the substance, without reference to the precise mechanism of such decrease. For example, a substance can be an inhibitor of enzyme activity by competitive, non-competitive, allosteric or other type of enzyme inhibition, by decreasing expression of the enzyme, or other direct or indirect mechanisms. Co-administration of a given drug with an inhibitor may decrease the rate of metabolism of that drug through the metabolic pathway listed.

By way of example only, in some embodiments, “synaptic dopamine” can be measured non-invasively as follows. Subjects receive an oral dose of a VMAT2 inhibitor before administration of [¹¹C]-PHNO, with PET imaging occurring around the time of maximal VMAT2 inhibitor plasma concentration. The binding potential relative to the non-displaceable binding (BP_{ND}) in the putamen, caudate, and ventral striatum, is used as the primary endpoint. The cerebellum is used as the reference region to estimate the regional BP_{ND}. The measured increases in [¹¹C]-PHNO BP_{ND} relative to baseline (ΔBP_{ND}) correspond to decreases in synaptic dopamine following VMAT2 inhibitor administration. Other radioligands can be used. Alternatively, in some embodiments, indirect measurements of synaptic dopamine can be achieved via invasive techniques such as methods described in Huang M., et al., Pharmacology, Biochemistry and Behavior 190 (2020) 172872; and Bosche S. L., et al., Frontiers in Neuroscience, November 2021|Volume 15|Article 728092.

“Image scanning” a subject refers to having the subject undergo scanning, e.g., positron emission tomography (PET) and/or computed tomography (CT) scanning.

The present application also includes pharmaceutically acceptable salts of the compounds described herein (e.g., pharmaceutically acceptable salts of VMAT2 inhibitors as described herein). As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As will be appreciated by those skilled in the art, the compounds provided herein, including salts and stereoisomers thereof, can be prepared using known organic synthesis techniques and can be synthesized according to any of numerous possible synthetic routes.

As used herein, “valbenazine” may be referred to as (S)-2-amino-3-methyl-butyric acid (2R, 3R,11bR)-3-isobutyl-9,10-dimethoxy-1,3,4,6,7,11b-hexahydro-2H-pyrido[2,1-a]isoquinolin-2-yl ester; or as L-Valine, (2R,3R,11bR)-1,3,4,6,7,11b-hexahydro-9,10-dimethoxy-3-(2-methylpropyl)-2H-benzo[a]quinolizin-2-yl ester or as NBI-98854.

As used herein, “NBI-98782” or “(+)-α-HTBZ” refers to the compound which is an active metabolite of valbenazine having the structure:

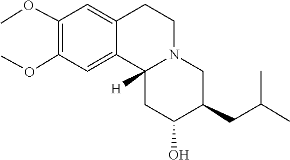 [SMILES: COc1cc2c(cc1OC)[C@H]1C[C@@H](O)[C@H](CC(C)C)CN1CC2]

(+)-α-HTBZ may be referred to as (2R, 3R, 11bR) or as (+)-α-DHTBZ or as (+)-α-HTBZ or as R,R,R-DHTBZ or as (+)-α-3-isobutyl-9,10-dimethoxy-1,3,4,6,7,11b-hexahydro-2H-pyrido[2,1-a]isoquinolin-2-ol; or as (2R, 3R,11bR)-3-isobutyl-9,10-dimethoxy-1,3,4,6,7,11b-hexahydro-2H-pyrido[2,1-a]isoquinolin-2-ol.

As used herein, the term “NBI-750142” refers to the compound having the following structure:

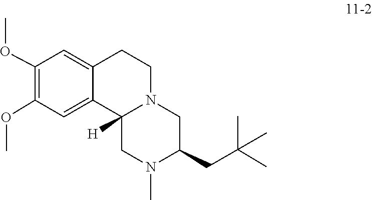 [SMILES: COc1cc2c(cc1OC)[C@H]1CN(C)[C@H](CC(C)(C)C)CN1CC2]

See e.g., U.S. Pat. No. 11,040,970, the disclosure of which is incorporated herein by reference in its entirety.

The compounds (e.g., VMAT2 inhibitors) and pharmaceutical compositions described herein (e.g., pharmaceutical compositions comprising a VMAT2 inhibitor) can inhibit the activity of VMAT2 in a subject. Compounds which inhibit VMAT2 are useful in providing a means of treating neurological and psychiatric diseases and disorders.

Example neurological and psychiatric diseases and disorders associated with VMAT2 include, but are not limited to hyperkinetic disorders (i.e., hyperkinetic movement disorder or “hyperkinesias”). Additional examples of diseases or disorders treatable by VMAT2 inhibition can be found, for example, in U.S. Pat. Nos. 10,065,952, 10,857,137, 10,857,148, 10,912,771, 10,952,997, 10,993,941, 11,026,931, and 11,040,029, the disclosures of which are incorporated herein by reference in their entireties; and U.S. Publication Nos.: 20200078352 and 20200397779, the disclosures of which are incorporated herein by reference in their entireties.

As used herein, “hyperkinetic disorder” or “hyperkinetic movement disorder” or “hyperkinesias” refers to disorders or diseases characterized by excessive, abnormal, involuntary movements. These neurological disorders include tremor, dystonia, myoclonus, athetosis, Huntington's disease, tardive dyskinesia, Tourette syndrome, dystonia, hemiballismus, chorea, senile chorea, or tics.

As used herein, “tardive syndrome” encompasses but is not limited to tardive dyskinesia, tardive dystonia, tardive akathisia, tardive tics, myoclonus, tremor and withdrawal-emergent syndrome. Tardive dyskinesia is characterized by rapid, repetitive, stereotypic, involuntary movements of the face, limbs, or trunk.

As used herein, “administering” refers to the process of introducing a composition or dosage form into a patient via an art-recognized means of introduction.

As used herein the term “disorder” is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not a free base form of a VMAT2 inhibitor, the quantity is the molar equivalent to the corresponding amount of the VMAT2 inhibitor free base form. For example, often a drug is packaged in a pharmaceutically acceptable salt form and the dosage for strength refers to the mass of the molar equivalent of the corresponding free base. As used herein “dose” administered is used interchangeably with “amount” administered.

As used herein, “effective amount” and “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “patient” or “subject” means a mammal, including a human, for whom or which therapy is desired, and generally refers to the recipient of the therapy.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

### Pharmaceutical Compositions

Also provided herein is a pharmaceutical composition for use in treating neurological or psychiatric diseases or disorders, comprising the VMAT2 inhibitor as an active pharmaceutical ingredient, in combination with one or more pharmaceutically acceptable carriers or excipients.

The choice of excipient, to a large extent, depends on factors, such as the particular mode of administration, the effect of the excipient on the solubility and stability of the active ingredient, and the nature of the dosage form.

The pharmaceutical compositions provided herein may be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered alone, or in combination with one or more other compounds provided herein, one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, PA); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as com starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, com oil, and soybean oil; zinc stearate; ethyl oleate; ethyl laureate; agar; starch; lycopodium; silica or silica gels, such as AEROSIL®200 (W.R. Grace Co., Baltimore, MD) and CAB-0-SIL® (Cabot Co. of Boston, MA); and mixtures thereof. The pharmaceutical compositions provided herein may contain about 0.1 to about 5% by weight of a lubricant. Suitable glidants include colloidal silicon dioxide, CAB-0-SIL® (Cabot Co. of Boston, MA), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation. The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms. The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H₂-receptor antagonists.

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

### Parenteral Administration

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science.

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, com oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl phydroxybenzates, thimerosal, benzalkonium chloride, benzethonium chloride, methyl- and propylparabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including alpha-cyclodextrin, beta-cyclodextrin, hydroxypropyl-beta-cyclodextrin, sulfobutylether-beta-cyclodextrin, and sulfobutylether 7-beta-cyclodextrin (CAPTISOL®, CyDex, Lenexa, KS).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In certain embodiments, the pharmaceutical compositions are provided as ready-to-use sterile solutions. In certain embodiments, the pharmaceutical compositions are provided as sterile dry soluble products, including lyophilized powders and hypodermic tablets, to be reconstituted with a vehicle prior to use. In certain embodiments, the pharmaceutical compositions are provided as ready-to-use sterile suspensions. In certain embodiments, the pharmaceutical compositions are provided as sterile dry insoluble products to be reconstituted with a vehicle prior to use. In certain embodiments, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semisolid, or thixotropic liquid, for administration as an implanted depot. In certain embodiments, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### Topical Administration

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, CA), and BIOJECT™ (Bioject Medical Technologies Inc., Tualatin, OR).

The pharmaceutical compositions provided herein may be provided in the forms of ointments, creams, and gels. Suitable ointment vehicles include oleaginous or hydrocarbon bases, including such as lard, benzoinated lard, olive oil, cottonseed oil, and other oils, white petrolatum; emulsifiable or absorption bases, such as hydrophilic petrolatum, hydroxystearin sulfate, and anhydrous lanolin; water-removable bases, such as hydrophilic ointment; water-soluble ointment bases, including polyethylene glycols of varying molecular weight; emulsion bases, either water-in-oil (W/O) emulsions or oil-in-water (O/W) emulsions, including cetyl alcohol, glyceryl monostearate, lanolin, and stearic acid. These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids, and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as 50 micrometers or less, or 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as /-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

### Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms.

The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

The pharmaceutical compositions provided herein in a modified release dosage form may be fabricated using a matrix-controlled release device known to those skilled in the art.

In certain embodiments, the pharmaceutical compositions provided herein in a modified release dosage form is formulated using an erodible matrix device, which is water swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, NJ); poly(2-hydroxyethyl-methacrylate); polylactides; copolymers of L-glutamic acid and ethyl-L-glutamate; degradable lactic acidglycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In certain embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as camauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents are water-swellable hydrophilic polymers, which are also referred to as “osmopolymers” and “hydrogels”. Suitable osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, DE) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water, as disclosed in U.S. Pat. No. 5,798,119. Such hydrophobic but water-permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes. The delivery port(s) on the semipermeable membrane may be formed postcoating by mechanical or laser drilling. Delivery port(s) may also be formed in situ by erosion of a plug of water-soluble material or by rupture of a thinner portion of the membrane over an indentation in the core. In addition, delivery ports may be formed during coating process.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

The pharmaceutical compositions provided herein in a modified release dosage form may be fabricated a multiparticulate controlled release device, which comprises a multiplicity of particles, granules, or pellets, ranging from about 10 pm to about 3 mm, about 50 pm to about 2.5 mm, or from about 100 pm to 1 mm in diameter. Such multiparticulates may be made by the processes know to those skilled in the art, including wet- and dry-granulation, extrusion/spheronization, roller-compaction, melt-congealing, and by spray-coating seed cores.

Other excipients as described herein may be blended with the pharmaceutical compositions to aid in processing and forming the multiparticulates. The resulting particles may themselves constitute the multiparticulate device or may be coated by various film forming materials, such as enteric polymers, water-swellable, and water-soluble polymers. The multiparticulates can be further processed as a capsule or a tablet.

### Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems.

### Labeled Compounds

In some embodiments, the VMAT2 inhibitor provided herein is an isotopic variant of a VMAT2 inhibitor. As used herein, “isotopic variant” refers to a compound containing an unnatural proportion of an isotope at one or more of the atoms that constitute such a compound. In certain embodiments, an “isotopic variant” of a compound contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (14N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

In some embodiments, the present application provides radiolabeled compounds, and uses thereof. In some embodiments, a radiolabeled compound provided herein comprises at least one radioisotope selected from the group consisting of ¹¹C, ¹³N, ¹⁵O, ¹⁸F, ⁶⁸Ga, ⁸⁹Zr, ⁸²Rb, ¹²⁴I, and ¹³¹I. In some embodiments, the radioisotope is a positron emitter. As used herein the term “positron emitter” refers to a radioisotope wherein a proton is converted to a neutron, thereby releasing a positron and an electron neutrino. In some embodiments, the positron emitter is ¹¹C or ¹⁸F. In some embodiments, the radiolabeled compounds provided herein comprises at least one ¹⁸F radioisotope. In some embodiments, the radiolabeled compounds provided herein is suitable for use as an imaging agent. In some embodiments, the radiolabeled compounds provided herein is suitable for use as a positron emission tomography (PET) imaging agent.

### Kits

The present application further includes pharmaceutical kits useful, for example, in the treatment diseases and disorder referred to herein, which include one or more containers containing a pharmaceutical composition described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Abbreviations

The following abbreviations may be used throughout the present application.
ALT alanine aminotransferase
AST aspartate aminotransferase
BMI Body mass index
BP_{ND} Binding potential relative to non-displaceable compartment
CNS Central Nervous System
EC₅₀ 50% effective concentration
ECG Electrocardiogram
Eₘₐₓ Maximum effect
GGT gamma-glutamyl transferase
IM Intramuscular
i.v. Intravenous
mCi Millicurie
MRI Magnetic resonance imaging
NHP Non-human primate
PET Positron emission tomography
PK Pharmacokinetic
QT Electrocardiographic interval from the beginning of the QRS complex to the end of the T wave
QTcF QT interval corrected for heart rate using Fridericia's formula
RDA Recommended dietary allowances
ROI Region of interest
SAE Serious adverse event
SUV Standard uptake value
SUVr Standard uptake value ratio
TAC Time activity curves
UDS Urine drug screen
ULN Upper limit of normal
URF Upregulation factor
VMAT2 Vesicular monoamine transporter type 2
VOI Volume of interest

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1. VMAT2 Receptor Occupancy Studies in Non-Human Primate (NHP) Following Intravenous (i.v.) Administration of NBI-98782 Using [¹⁸F]AV-133 Positron Emission Tomography (PET)

The aim of this study was to assess the relationship between NBI-98782 dose/plasma concentration and VMAT2 occupancy with [¹⁸F]AV-133 using PET in non-human primate (NIP) brain. NBI-98782 (i.e., [+]-α-HTBZ; or (+)-α-3-isobutyl-9,10-dimethoxy-1,3,4,6,7,11b-hexahydro-2H-pyrido[2,1-a]isoquinolin-2-ol; or (2R, 3R,11bR)-3-isobutyl-9,10-dimethoxy-1,3,4,6,7,11b-hexahydro-2H-pyrido[2,1-a]isoquinolin-2-ol) is the active metabolite of valbenazine, which has been developed as a therapeutic to inhibit VMAT2 and reduce dopamine release at presynaptic nerve terminals. A radioligand, [¹⁸F]AV-133, has been used to image and quantify brain VMAT2 in vivo. PET studies in NHPs have been shown to provide useful information on brain access and target engagement by novel compounds. Such data can be used to demonstrate target engagement and index pharmacodynamic outcomes of novel CNS compounds, allowing for identification of critical dosage ranges, e.g., in clinical settings.

### [¹⁸F]AV-133 Labeling Protocol

[¹⁸F]AV-133 was prepared from the following radiosynthetic scheme:

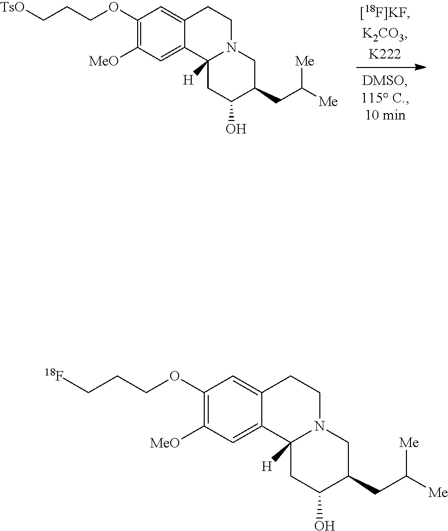 [SMILES: COc1cc2c(cc1OCCCOS(=O)(=O)c1ccc(C)cc1)CCN1C[C@@H](CC(C)C)[C@H](O)C[C@H]21 ; COc1cc2c(cc1OCCC[18F])CCN1C[C@@H](CC(C)C)[C@H](O)C[C@H]21]

A high performance liquid chromatography (HPLC) chromatogram demonstrated the purity of the final purified radiolabeled [¹⁸F]AV-133 after labeling.

### Preparation of the Dosing Formulations

[¹⁸F]AV-133 was formulated in ethanol and ascorbic acid in saline. NBI-98782 was dissolved in 0.9% sterile saline and the resulting solution was filtered through 0.2 μm sterile filter into a sterile, empty glass vial. Approximately 5 mL of formulated material was passed through the filter and discarded prior to collection of final dosing formulation to avoid potential loss during filtration. The solution was visually inspected and found to be clear and free of particulates. For all preparations, concentrations are reported as NBI-98782 free base using a correction factor of 1.73.

### Animal Model

Two female non-naïve cynomolgus monkeys (Macaca fascicularis) were used in this study. The animals were judged to be in good health before the start of the study. At study origination, animals A7701 (Tag ID A5028) and A7702 (Tag ID A5029) were 4.45 kg/4 years 11 months and 4.75 kg/3 years 10 months, respectively.

### In Vivo Imaging Study

The animals were imaged with [¹⁸F]AV-133 at baseline with vehicle (0.9% saline) and post NBI-98782 blockade on separate days. Vehicle and NBI-98782 were administered as an intravenous (i.v.) bolus followed by a constant infusion over 3 hours. [¹⁸F]AV-133 was injected as an i.v. bolus, 1 h after administration of vehicle/NBI98782. The design study is summarized in Table 1-1.

TABLE 1-1

| | | | | | Target |
|---|---|---|---|---|---|
| | | | | NBI- | NBI- |
| | | | | 98782 | 98782 |
| | | | NBI- | Infusion | Total |
| | | | 98782 | Over | Mass |
| Group & | | | Bolus | 3 Hours | Dose |
| Route | Animal | Condition | (mg/kg) | (mg/kg/3h) | (mg/kg) |
| 1 IV | A7701 & | Baseline, | n/a | n/a | n/a |
| | A7702 | vehicle | | | |
| 2 IV | A7701 | Blockade | 0.03 | 0.1 | 0.13 |
| 3 IV | A7702 | Blockade | 0.08 | 0.3 | 0.38 |
| 4 IV | A7701 | Blockade | 0.01 | 0.03 | 0.04 |
| 5 IV | A7702 | Blockade | 0.004 | 0.012 | 0.016 |
| 6 IV | A7701 | Blockade | 0.1 | 0.4 | 0.5 |
| 7 IV | A7702 | Blockade | 0.03 | 0.1 | 0.13 |

### Dose Administration

Each animal was weighed on the day of dose administration. Two intravenous lines were placed and used for administration of (1) the radiopharmaceutical [¹⁸F]AV-133) and (2) the test article (NBI-98782) or vehicle (0.9% saline). See Table 1-2 and Table 1-3 for details.

Monkeys were fasted overnight before each individual PET scan. Prior to administration of the NBI-98782 or vehicle, the animals were sedated with ketamine, 5-10 mg/kg IM. The animals were intubated with an endotracheal tube for continued delivery of isoflurane, to effect, for anesthesia maintenance. Hydration was maintained with lactated Ringer's solution (LRS), 3-5 mL/kg/h IV. Isoflurane and fluid levels were adjusted during the course of a study to maintain an anesthesia.

Body temperature was maintained using a heated water blanket. Vital signs, including heart rate, respiration rate, oxygen saturation and body temperature, were monitored at least every 10 to 15 min throughout the period in which the monkey was under anesthesia.

### Image Acquisition and Processing

Dynamic data were acquired in the MicroPET Focus-220 camera (Siemens Microsystems, Knoxville, TN). Emission data were collected for 120 min post-injection and reconstructed into a series of 33 frames and all standard corrections were applied: normalization, random, scatter and attenuation (via CT scan). Dynamic brain PET images were transferred and analyzed using the image processing PMOD software package v3.802 (PMOD Technologies, Zurich, Switzerland). The PET images were rigidly aligned to a previously acquired animal's brain T1 MRI and then spatially normalized to a common cynomolgus MRI template for anatomical brain region definition.

### Analysis Methods

### Assessing NBI-98782 Concentration

During each scan, whole blood PK samples were collected (in K2 EDTA tubes) at pre-dose (−5 min), and post-dose at 4 (approximately 1 min after completion of bolus), 30, 60 (just prior to [¹⁸F]AV-133 radiopharmaceutical administration), 90, 120, 150, 180 (end of scan) minutes after test article administration. PK samples were approximately 1 mL each (8 mL total, including pre-dose sample). PK sample collection times were relative to start of NBI-98782 bolus administration.

NBI-98782 concentration in cynomolgus monkey plasma samples was analyzed using validated LC-MS/MS method. The NBI-98782 assay has a quantified range from 0.50 to 500 ng/mL, with a dilution integrity demonstrated up to 12,500 ng/mL. Plasma samples were processed using liquid-liquid extraction with the additional of a stable isotopic labeled internal standard ¹³C-83198. Processed samples were chromatographically separated on a C18 reversed phase column and coupled to a Sciex API 4000 triple quadrupole mass spectrometer for detection. NBI-98782 mean concentrations are presented in Table 1-6.

### Non-GLP Determination of NBI-98782 in Cynomolgus Monkey Stabilized K2-EDTA Plasma in a Cyno PET Imaging Study by LC-MS-MS

This method was validated for a range of 0.500 to 500 ng/mL for NBI-98782 based on the analysis of 100 μL of Cynomolgus monkey plasma by LC-MS-MS. Quantitation was performed using a weighted 1/×2 linear least squares regression analysis generated from calibration standards prepared on the day of extraction. The liquid-liquid extraction began with the addition of internal standard solution (10.0 μL of approximately 400 ng/mL of [¹³C]-radiolabeled [¹³C]-NBI-98854 (i.e., [¹³C]-valbenazine) and [¹³C]-NBI-83198 (a racemic mixture of two dihydrotetrabenazine enantiomers). After adding 50.0 μL of sodium carbonate solution (0.2 M) and 2.00 mL of methyl tert-butyl ether to all samples, the tubes were sealed, vortexed, and centrifuged. Subsequently, the organic layer was transferred to clean tubes and evaporated to dryness. The samples were then reconstituted with 150 μL of reconstitution solution (water/methanol/acetic acid; 80:20:0.2), sealed, vortexed, sonicated and centrifuged. The reconstituted extract was then transferred into a 96-well plate. Chromatographic separation was achieved with a HPLC Kinetex C18 column (2.6 μm, 50×2.1 mm) and gradient elution using mobile phase A (water/ammonium acetate solution/acetic acid; 1000:5:1) and mobile phase B (2-propanol/methanol/ammonium acetate solution/acetic acid; 300:700:5:1). A triple quadrupole mass spectrometer (Sciex API 4000) equipped with TurboIonSpray source was used in the positive ion mode. Quantitation was based on multiple reaction monitoring (MR) of the m/z transitions for NBI-98782 and [¹³C]-NBI-83198.

### Visualizing Tissue Uptake

Volumes of interest (VOIs) defined in the cynomolgus template space (see e.g., Ballanger et al. Neuroimage, 2013, 77:26-43) were applied to the spatially normalized PET images to compute time-activity curves (TACs, kBq/cm³) for caudate (0.96 cm³), putamen (1.34 cm³) and occipital (8.61 cm³). TACs and images (averaged between 90-120 min) were presented in Standard Uptake Value (SUV) units (g/mL) normalized by the weight of the animal and the injected dose

### Image Quantification

Non-invasive Logan graphical analysis (start time of fit, t*=30 min) was used to estimate regional non-displaceable binding potential (BPND) using the TACs in the caudate and putamen, with the cerebellar cortex as the reference TAC. BPND was calculated in the in the caudate and putamen across baseline and NBI-98782 scans. Target occupancy was calculated according to the Equation 2-1.

$$Occ ( \% ) = ( {BP}_{ND}^{Baseline} - {BP}_{ND}^{NBI - 98782} ) / {BP}_{ND}^{Baseline} \times 100. \qquad Equation 2 - 1$$

The relationship between the occupancy at VMAT2, averaged between caudate and putamen (i.e., striatum), and the administered dose or the average plasma concentration of NBI-98782 during the time of the scan was determined with a single specific binding site Eₘₐₓ model with a fixed maximum occupancy of 100%, according to Equation 2-2,

$$Occ = \frac{100 \% \times {C}^{h}}{{EC}_{50}^{h} + {C}^{h}} . \qquad Equation 2 - 2$$

where h is the Hill slope (h=1.0) and C is the mean of observed plasma concentrations during the scanning interval (60 to 180 minutes post-dose).

### Results

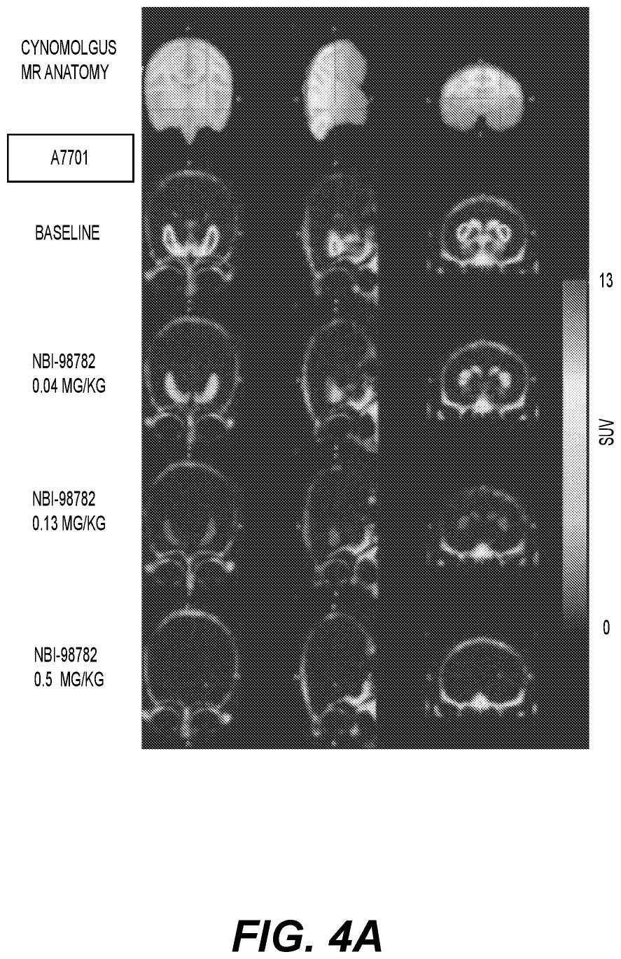

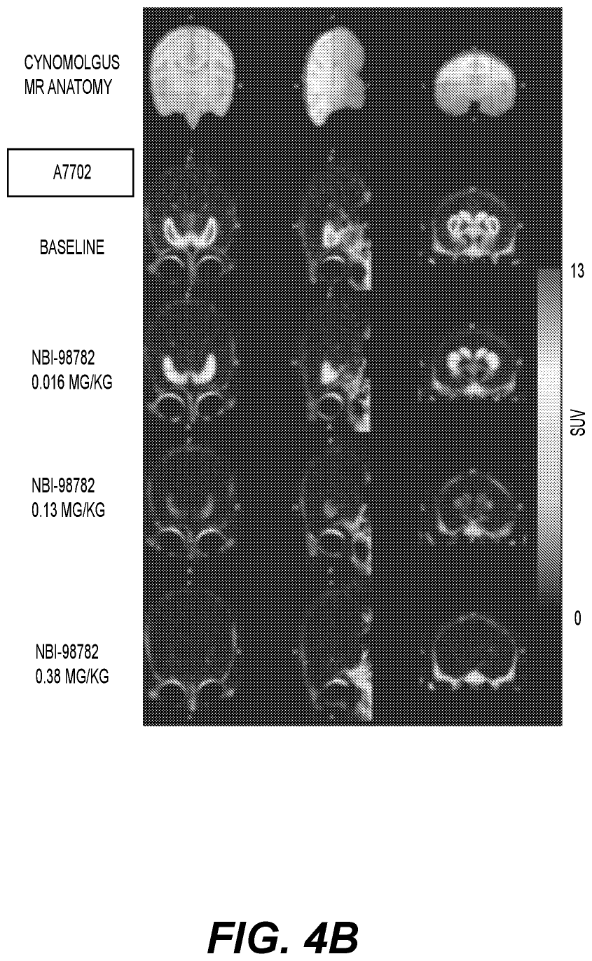

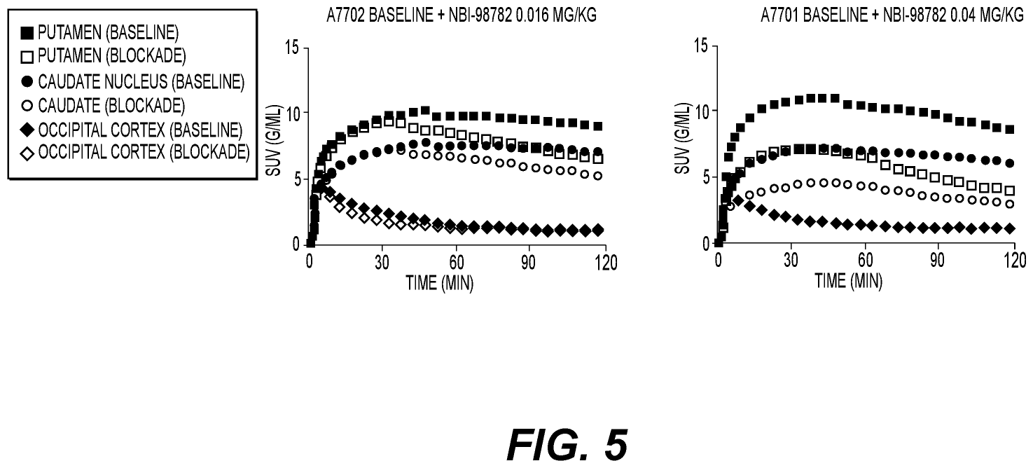

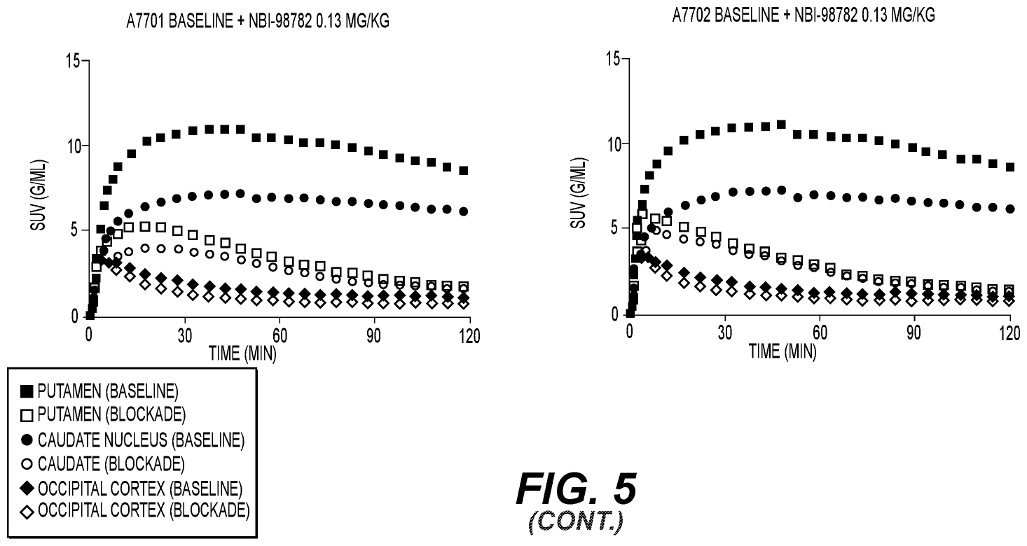

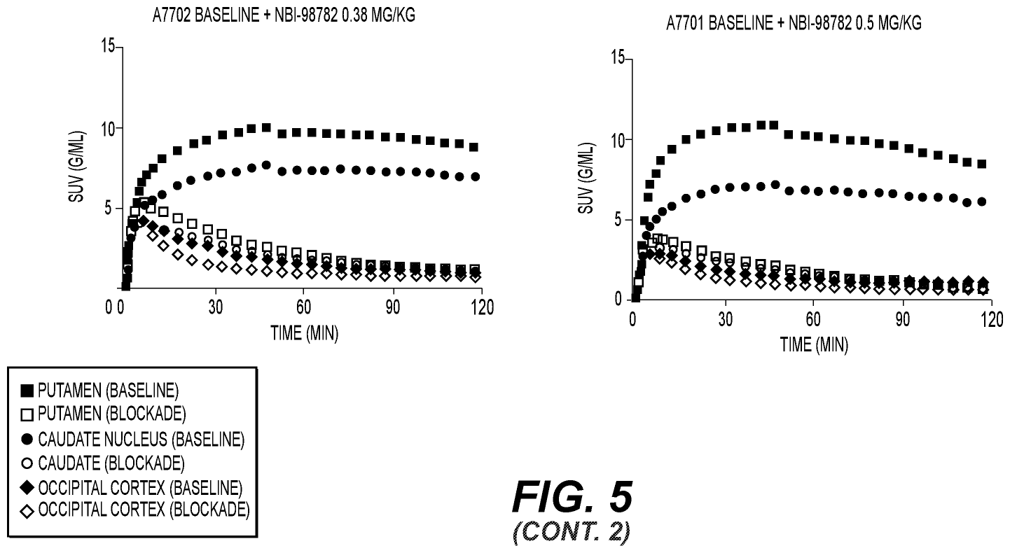

Two female cynomolgus monkeys were imaged with [¹⁸F]AV-133 (5.5±0.9 mCi) at baseline and after administration of NBI-98782 to examine the relationship of average total NBI-98782 concentration during the time of the scan (mean concentration from 60-180 minutes post-NBI-98782 dose) and VMAT2 occupancy in the striatum (occupancy averaged from caudate and putamen). Table 1-2 and Table 1-3 show [¹⁸F]AV-133 and NBI-98782 injection information for each evaluation. Across evaluations, [¹⁸F]AV-133 SUV images are shown in FIG. 4 and TACs are shown in FIG. 5.

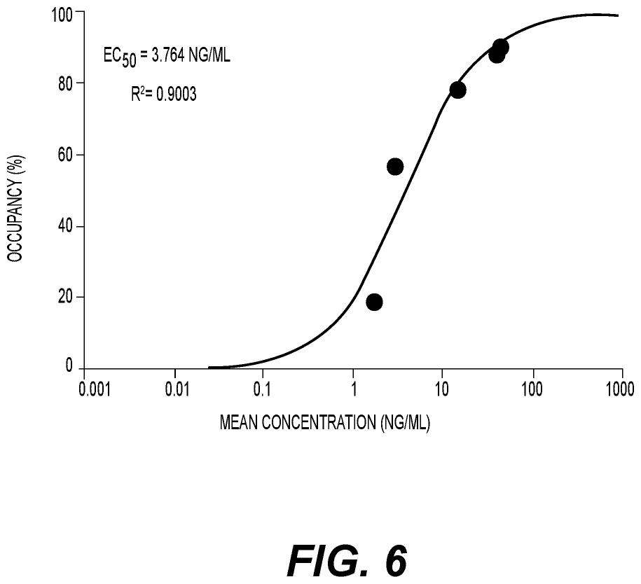

At baseline, there was high uptake of [¹⁸F]AV-133 in the caudate and putamen relative to the rest of the brain. A dose- and concentration-dependent decrease in [¹⁸F]AV-133 binding was observed post administration of NBI-98782. BPND and occupancy are provided in Table 1-4 for animal A7701 and Table 1-5 for animal A7702. Total NBI-98782 concentration and [¹⁸F]AV-133 striatal occupancy are provided in Table 1-6. The relationship of occupancy of NBI-98782 with total plasma concentration is shown in FIG. 6, where the 50% effective total concentration (EC₅₀) was 3.8 ng/mL with a 95% confidence interval between 1.9 ng/mL and 7.5 ng/mL.

TABLE 1-2
[¹⁸F]AV-133 Radiopharmaceutical (IV) Administration Records

| | | | | [¹⁸F]MNI-1138 | |
|---|---|---|---|---|---|
| Animal | | Weight | Injection | Activity | Volume |
| ID | Group | (kg) | Date | (mCi) | (mL) |
| 7701 | 1 | 4.45 | Jan. 14, 2020 | 5.1 | 1.1 |
| 7702 | 1 | 4.75 | Jan. 15, 2020 | 5.9 | 1.28 |
| 7701 | 2 | 4.3 | Feb. 4, 2020 | 5.8 | 2.37 |
| 7702 | 3 | 5.1 | Mar. 13, 2020 | 5.5 | 2.46 |
| 7701 | 4 | 4.5 | Mar. 13, 2020 | 5.4 | 5.92 |
| 7702 | 5 | 4.8 | Mar. 31, 2020 | 5.3 | 1.8 |
| 7701 | 6 | 4.35 | Apr. 17, 2020 | 5.9 | 2.21 |
| 7702 | 7 | 4.85 | Apr. 17, 2020 | 6.3 | 5.19 |

TABLE 1-3
NBI-98782 (IV) Administration Records

| | | | | NBI-98782 | | | | |
|---|---|---|---|---|---|---|---|---|
| | | | | Bolus | Bolus | Infusion | Infusion | Total |
| Animal | | Weight | Injection | Conc. | Volume | Conc. | Volume | Dose |
| ID | Group | (kg) | Date | (mg/mL) | (mL) | (mg/mL) | (mg/mL) | (mg/kg) |
| 7701 | 1 | 4.45 | Jan. 14, 2020 | n/a | 0.44 | n/a | 1.47 | n/a |
| 7702 | 1 | 4.75 | Jan. 15, 2020 | n/a | 0.44 | n/a | 1.48 | n/a |
| 7701 | 2 | 4.3 | Feb. 4, 2020 | 0.29 | 0.44 | 0.29 | 1.47 | 0.13 |
| 7702 | 3 | 5.1 | Mar. 13, 2020 | 0.906 | 0.45 | 1.02 | 1.51 | 0.38 |
| 7701 | 4 | 4.5 | Mar. 13, 2020 | 0.1 | 0.45 | 0.09 | 1.50 | 0.04 |
| 7702 | 5 | 4.8 | Mar. 31, 2020 | 0.0427 | 0.45 | 0.0383 | 1.47 | 0.016 |
| 7701 | 6 | 4.35 | Apr. 17, 2020 | 0.966 | 0.45 | 1.16 | 1.47 | 0.5 |
| 7702 | 7 | 4.85 | Apr. 17, 2020 | 0.323 | 0.45 | 0.323 | 1.47 | 0.13 |

n/a = not applicable, vehicle only

TABLE 1-4
[¹⁸F]AV-133 BPND and occupancy (% Occ) in animal A7701

| | | BP_{ND} | BP_{ND} | BP_{ND} | | | |
|---|---|---|---|---|---|---|---|
| | | NBI- | NBI- | NBI- | | | |
| | | 98782 | 98782 | 98782 | % Occ | % Occ | % Occ |
| | BP_{ND} | 0.04 | 0.13 | 0.5 | 0.04 | 0.13 | 0.5 |
| Region | Baseline | mg/kg | mg/kg | mg/kg | mg/kg | mg/kg | mg/kg |
| Caudate | 4.16 | 1.70 | 1.34 | 0.44 | 59.1 | 67.9 | 89.4 |
| Putamen | 6.37 | 2.86 | 1.86 | 0.66 | 55.2 | 70.8 | 89.6 |

TABLE 1-5
[¹⁸F]AV-133 BPND and occupancy (% Occ) in animal A7702

| | | BP_{ND} | BP_{ND} | BP_{ND} | | | |
|---|---|---|---|---|---|---|---|
| | | NBI- | NBI- | NBI- | | | |
| | | 98782 | 98782 | 98782 | % Occ | Occ | % Occ |
| | BP_{ND} | 0.016 | 0.13 | 0.38 | 0.016 | 0.13 | 0.38 |
| Region | Baseline | mg/kg | mg/kg | mg/kg | mg/kg | mg/kg | mg/kg |
| Caudate | 4.83 | 3.93 | 1.09 | 0.52 | 18.7 | 77.5 | 89.3 |
| Putamen | 6.33 | 5.17 | 1.33 | 0.86 | 18.3 | 79.1 | 89.4 |

TABLE 1-6
Mean Plasma NBI-98782 Concentrations Throughout the PET Scan & Target Occupancy During PET Scan

| Animal | A7701 | | | A7702 | | |
|---|---|---|---|---|---|---|
| Dose Date | Feb. 4, 2020 | Mar. 13, 2020 | Apr. 17, 2020 | Mar. 13, 2020 | Mar. 31, 2020 | Apr. 17, 2020 |
| Total Mass | 0.13 | 0.04 | 0.5 | 0.38 | 0.016 | 0.13 |
| Dose (mg/kg) | | | | | | |
| Mean ng/mL | n/a** | 2.9 | 44.5 | 39.9 | 1.7 | 14.9 |
| NBI-98782 | | | | | | |
| Concentration | | | | | | |
| during PET | | | | | | |
| Scan (60-180 | | | | | | |
| min) | | | | | | |
| Target | 69% | 57% | 90% | 88% | 19% | 78% |
| Occupancy, | | | | | | |
| Mean of | | | | | | |
| Putamen and | | | | | | |
| Caudate | | | | | | |

*BLQ = <0.5 ng/mL
**Excluded datapoint for 0.13 mg/kg dose in animal A7701 due to variability in plasma measurements.

### Conclusions

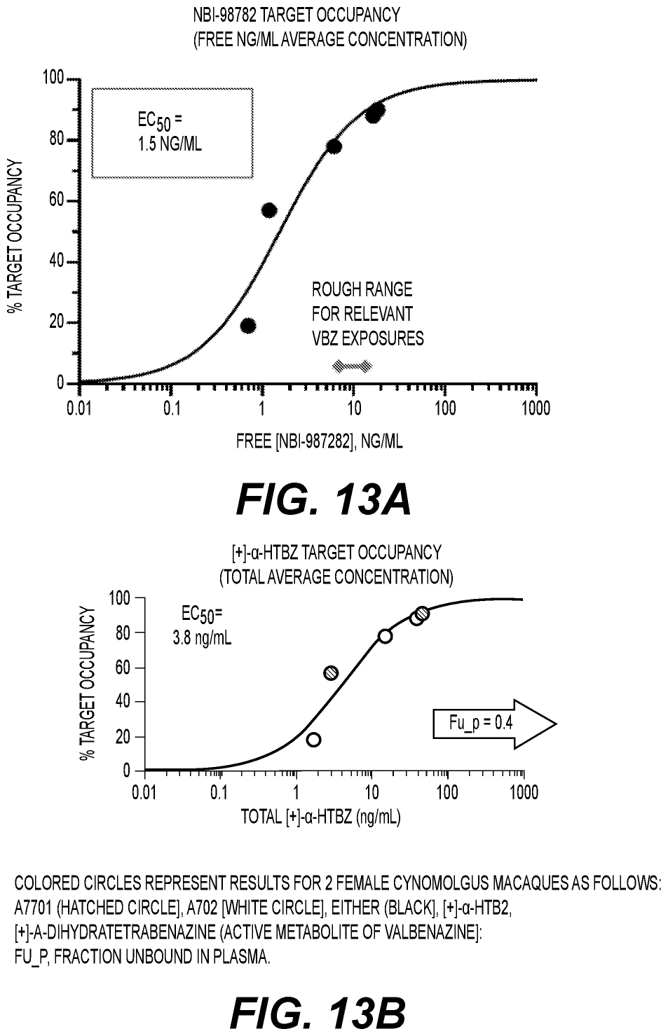

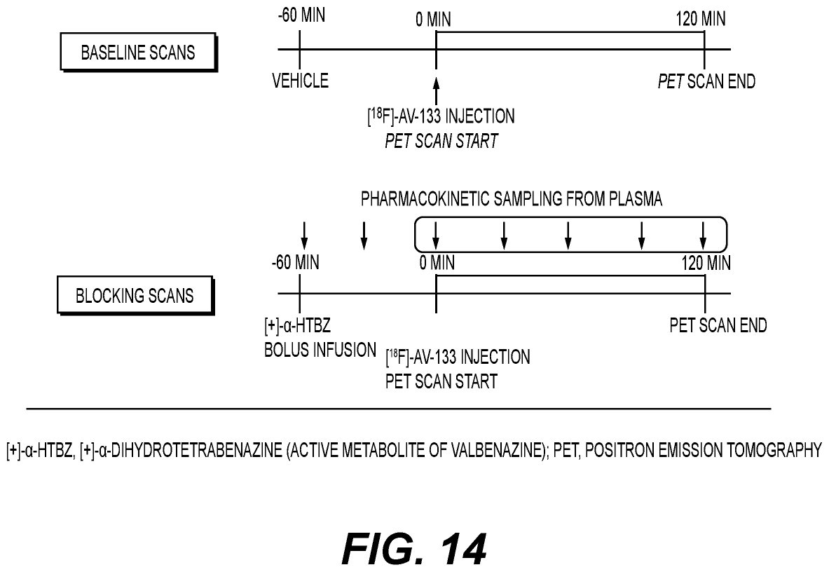

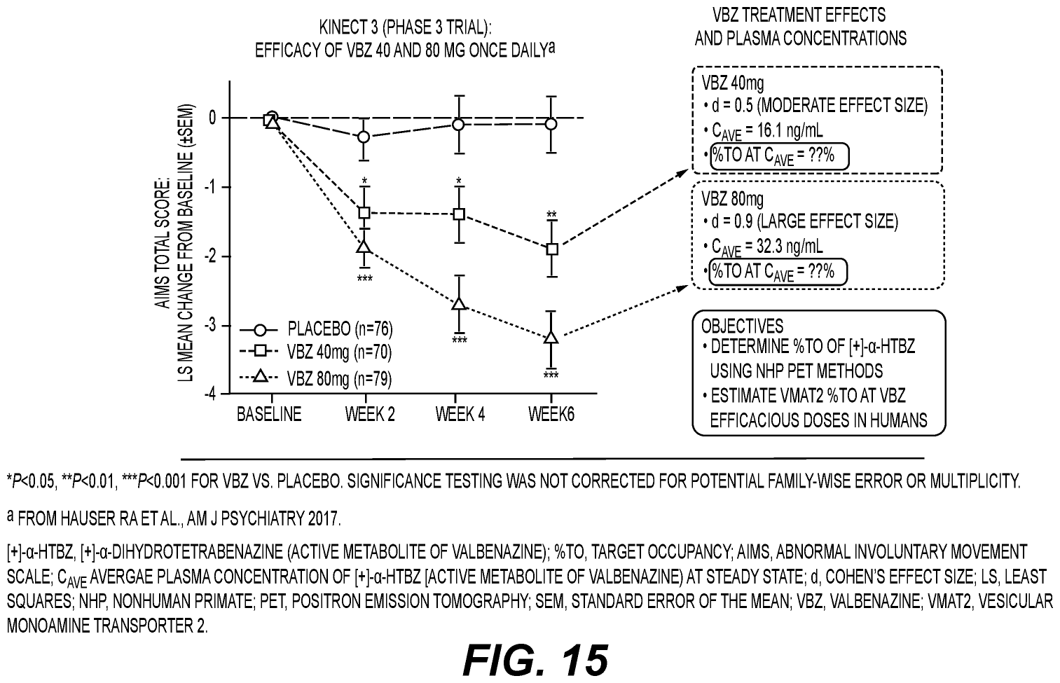

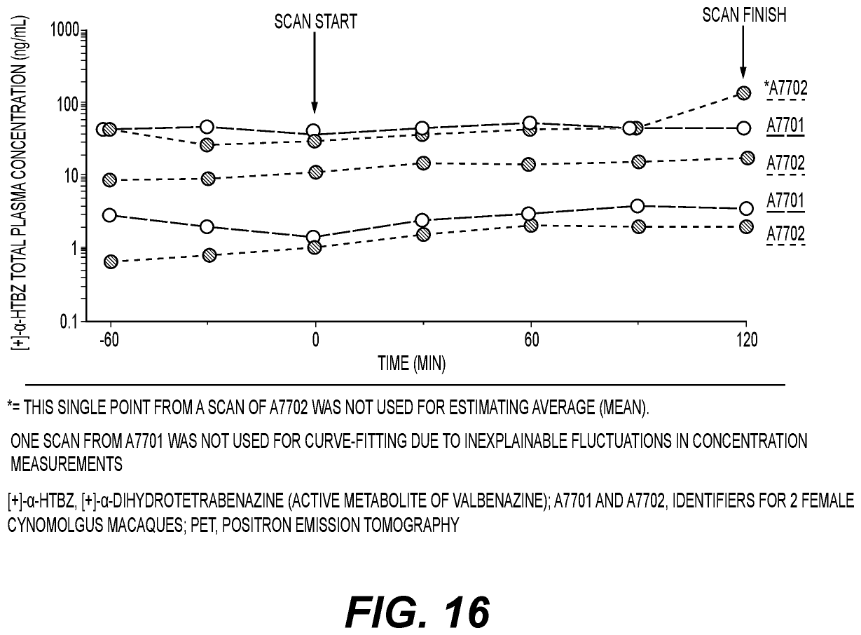

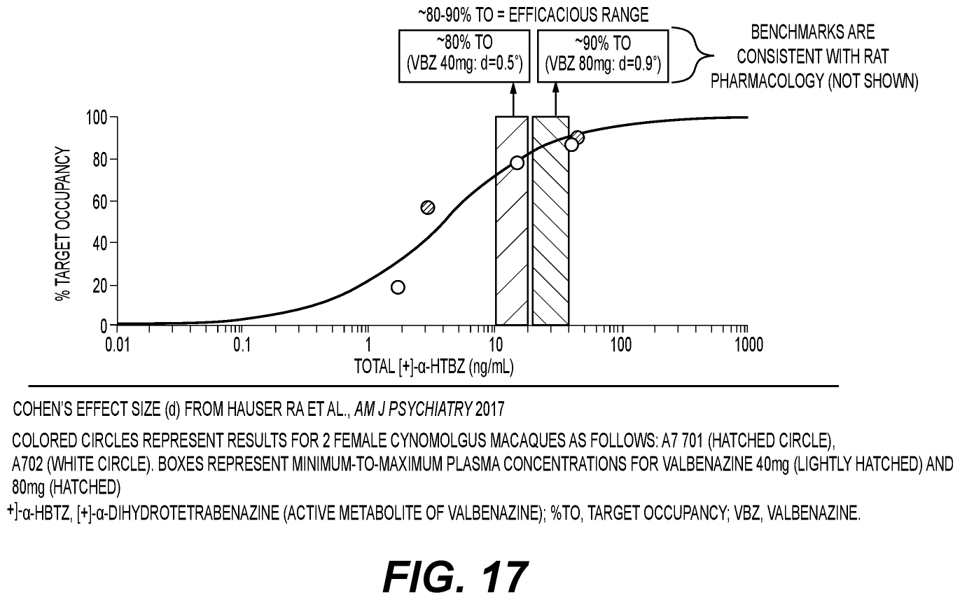

As measured by [¹⁸F]AV-133 PET imaging in NHP brain, NBI-98782 striatal occupancy of VMAT2 was concentration-dependent. Using an Eₘₐₓ model with a Hill slope of 1.0 and fixed maximum occupancy of 100%, the total plasma NBI-98782 concentration EC₅₀ for VMAT2 occupancy was 3.8 ng/mL (95% CI. 1.9 ng/mL and 7.5 ng/mL). Using cynomolgus monkey PPB data, the free (unbound) EC₅₀ was 1.5 ng/mL (see e.g. FIG. 13).

Using the two valbenazine doses that demonstrated efficacy in a Phase 3 trial of tardive dyskinesia (40 mg and 80 mg), along with the known pharmacokinetic properties of valbenazine, the PET methods described above were applied to estimate VMAT2 target occupancy (% TO) at therapeutic levels of valbenazine for TD treatment. Free plasma concentrations of NBI-98782 at steady-state in humans following once-daily dosing with valbenazine at 40 mg, 60 mg, or 80 mg were estimated from total plasma concentrations of NBI-98782 and human PPB data. The free EC₅₀ derived from NHP PET was then used to estimate % TO in humans for maximum, average, and minimum NBI-98782 plasma concentrations at steady state (Cₘₐₓ, Cₐᵥₑ, C_{trough}) based on clinically efficacious doses of valbenazine.

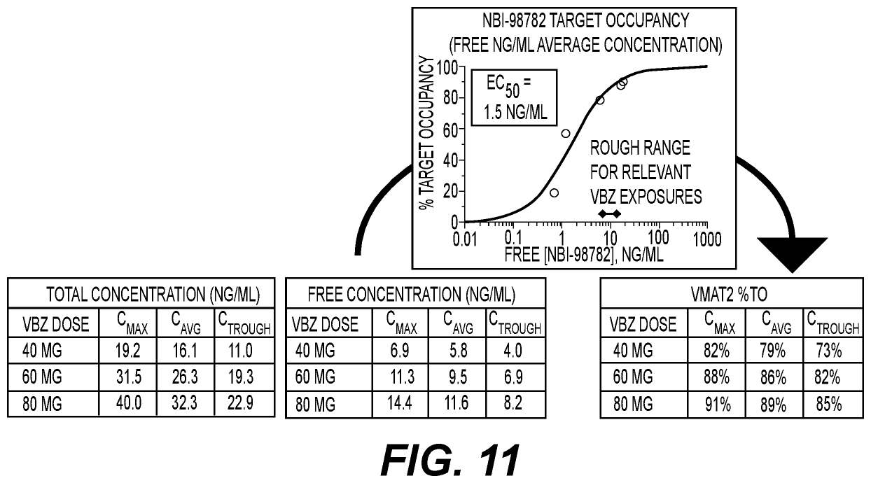

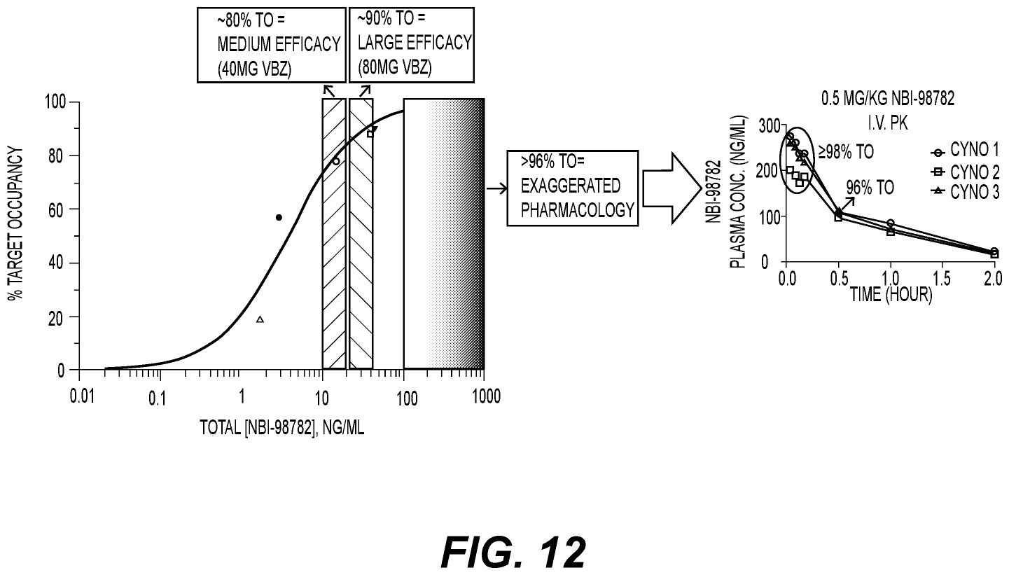

The EC₅₀ data was applied to human PK data at steady state, and indicated maintenance of VMAT2 target occupancy with once-daily valbenazine dosing as follows: 40 mg (73% to 82%); 60 mg (82% to 88%); 80 mg (85% to 91%), as shown in FIG. 11. At doses demonstrated to be clinically effective in the treatment of tardive dyskinesia, valbenazine is estimated to maintain a high VMAT2 occupancy (≥73%) throughout each 24-hour period (see FIG. 11). Relevant target occupancy (˜80% for Cₐᵥₑ) was achieved for valbenazine 40 mg once-daily, which is the recommended initiation dose for all patients with TD (and the lowest approved dose), indicating a potential biological effect from the beginning of treatment. At estimated 96%-98% VMAT2 target occupancy in cynomolgus monkey (see FIG. 12), there were clinical observations that were captured ˜30 min to 1 h post-dosing that included partially closed eyes (ptosis) (in all monkeys), decreased activity (in ⅔ monkeys), and salivation (in 1 monkey).

Based on the larger effect size for valbenazine 80 mg, versus valbenazine 40 mg, observed in the Phase 3 clinical trial, results from this study suggest that a high sustained occupancy (≥85%) and inhibition of VMAT2 underpins maximal efficacy for TD. Because adverse effects in monkey were observed at occupancies >95%, these results suggest once daily dosing of valbenazine 80 mg may be pharmacologically optimized for maximizing efficacy (e.g., by exceeding and maintaining 85% TO) while avoiding adverse effects (by staying below roughly 95% TO). The process described herein has matched the clinical efficacy of valbenazine with % TO estimations. These values, as summarized in FIG. 12, could then serve as benchmarks for comparing against any tetrabenazine-analog VMAT2 inhibitor. Although these benchmarks are defined by efficacy in treating TD, it is proposed the benchmarks would serve as a suitable “base case” for exploring VMAT2 inhibitor efficacy in any therapeutic area.

### Example 2. VMAT2 Receptor Occupancy Studies in Non-Human Primate (NHP) Following Intravenous (i.v.) Administration of NBI-750142 Using [¹⁸F]AV-133 Positron Emission Tomography (PET)

The aim of this study was to assess the relationship between NBI-750142 dose/plasma concentration and VMAT2 occupancy with [¹⁸F]AV-133 using PET in non-human primate (NIP) brain. A radioligand, [¹⁸F]AV-133, has been used to image and quantify brain VMAT2 in vivo. PET studies in NHPs have been shown to provide useful information on brain access and target engagement by novel compounds; such data can be used to demonstrate target engagement and index pharmacodynamic outcomes of novel CNS compounds, allowing for identification of critical dosage ranges, e.g., in clinical settings.

### [¹⁸F]AV-133 Labeling Protocol

[¹⁸F]AV-133 was prepared according to the following radiosynthetic scheme:

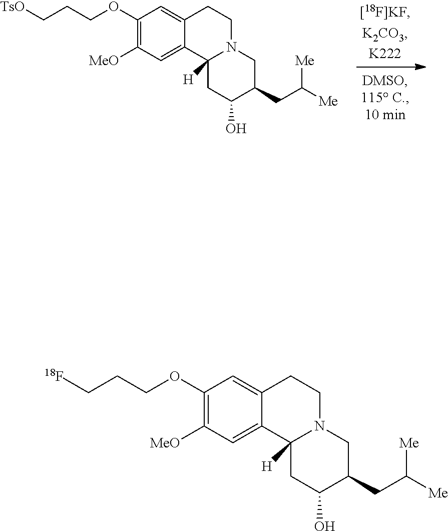 [SMILES: COc1cc2c(cc1OCCCOS(=O)(=O)c1ccc(C)cc1)CCN1C[C@@H](CC(C)C)[C@H](O)C[C@H]21 ; COc1cc2c(cc1OCCC[18F])CCN1C[C@@H](CC(C)C)[C@H](O)C[C@H]21]

A high performance liquid chromatography (HPLC) chromatogram demonstrated the purity of the final purified radiolabeled [¹⁸F]AV-133 after labeling.

### Preparation of the Dosing Formulations

[¹⁸F]AV-133 was formulated in saline, sodium ascorbate (4.66 mg/mL), and ethanol ≤10%. NBI-750142 was dissolved in 0.9% USP-grade saline and the resulting solution was filtered through 0.2-μm sterile filter into a sterile, empty glass vial. The solution was visually inspected and found to be clear and free of visible particulates. For all preparations, concentrations are reported as NBI-750142 free base using a correction factor of 1.59.

### Animal Model

Three non-naïve cynomolgus monkeys (Macaca fascicularis) were used in this study. The animals were judged to be in good health before the start of the study. At study origination the animals were 7 (female, EC865), 8 (male, GC786) and 9 (female, LC206) years old and weighed 4.2, 7.0, and 5.4 kg, respectively.

### In Vivo Imaging Study

The animals were imaged with [¹⁸F]AV-133 at baseline with vehicle (0.9% saline) and post NBI-750142 blockade on separate days. Vehicle and NBI-750142 were administered as an intravenous (i.v.) bolus followed by a constant infusion over 3 hours. [¹⁸F]AV-133 was injected as an i.v. bolus, 1 h after administration of vehicle/NBI-750142. The design study is summarized below Table 2-1.

TABLE 2-1
Study Design Summary

| | | | Target | | NBI- |
|---|---|---|---|---|---|
| | | | NBI- | | 750142 |
| | | | 750142 | NBI- | Infusion |
| | | | Total Mass | 750142 | Over |
| Group & | No. | | Dose | Bolus | 3 Hours |
| Route | Animals | Condition | (mg/kg) | (mg/kg) | (mg/kg) |
| 1 IV | 3 | Baseline | n/a | n/a | n/a |
| | | (Vehicle, | | | |
| | | saline) | | | |
| 2 IV | 1 | NBI-750142 | 0.026 | 0.01 | 0.016 |
| | | blockade | | | |
| 3 IV | 1 | NBI-750142 | 0.084 | 0.033 | 0.051 |
| | | blockade | | | |
| 4 IV | 2 | NBI-750142 | 0.26 | 0.1 | 0.16 |
| | | blockade | | | |
| 5 IV | 1 | NBI-750142 | 0.52 | 0.2 | 0.32 |
| | | blockade | | | |
| 6 IV | 1 | NBI-750142 | 0.78 | 0.3 | 0.48 |
| | | blockade | | | |

### Dose Administration

Each animal was weighed on the day of dose administration. Two intravenous lines were placed and used for administration of (1) the radiopharmaceutical [¹⁸F]AV-133) and (2) the test article (NBI-750142) or vehicle (0.900 saline). See Table 2-2 for details.

TABLE 2-2
[¹⁸F]AV-133 Radiopharmaceutical (IV) and NBI-750142 (IV) Injection Records

| | | | Date/ | [¹⁸F]MNI-1138 | | NBI-750142 | | |
|---|---|---|---|---|---|---|---|---|
| Animal | | Weight | Record | Activity | Volume | Conc. | Volume | Dose |
| ID | Group | (kg) | ID | (mCi) | (mL) | (mg/mL) | (mL) | (mg/kg) |
| LC206 | 1 | 5.5 | Aug. 11, 2018 | 2.92 | 7.8 | n/a | 9.9 | n/a |
| | | | 18A0078* | | | | | |
| GC786 | 1 | 6.9 | Apr. 1, 2019 | 3.70 | 9.7 | n/a | 10.1 | n/a |
| | | | 19A0010 | | | | | |
| EC865 | 1 | 4.2 | May 20, 2019 | 3.02 | 9.2 | n/a | 9.9 | n/a |
| | | | 19A0026 | | | | | |
| GC786 | 2 | 7 | Jun. 17, 2019 | 3.44 | 5.8 | 0.021 | 8.9 | 0.026 |
| | | | 19A0034 | | | | | |
| EC865 | 3 | 4.1 | Jun. 3, 2019 | 2.95 | 5.5 | 0.038 | 9.1 | 0.084 |
| | | | 19A0031 | | | | | |
| LC206 | 4 | 5.4 | Apr. 1, 2019 | 3.97 | 7.1 | 0.16 | 8.8 | 0.26 |
| | | | 19A0011 | | | | | |
| GC786 | 4 | 7.0 | Apr. 22, 2019 | 2.98 | 6.7 | 0.179 | 10.1 | 0.26 |
| | | | 19A0015 | | | | | |
| EC865 | 5 | 4.2 | Jun. 17, 2019 | 3.01 | 5.6 | 0.239 | 9.2 | 0.518 |
| | | | 19A0033 | | | | | |
| GC786 | 6 | 7.1 | May 6, 2019 | 3.96 | 8.0 | 0.63 | 8.8 | 0.78 |
| | | | 19A0020 | | | | | |

n/a = not applicable, vehicle only
*This baseline scan was part of a separate study, but it was used here for image analysis of subsequent scans in the same animal

Monkeys were fasted for 8-12 h before each individual PET scan. At least 60 minutes prior to administration of the NBI-750142 or vehicle, the animals were sedated with a combination of Aflaxan 2 mg/kg, dexmedetomidine 0.02 mg/kg, and midazolam 0.3 mg/kg, (intramuscular, IM), and transferred to the imaging suite. 1% lidocaine, 1-2 drops, was applied topically over larynx to control for intubation induced inflammation. The animals were immediately intubated with an endotracheal tube for continued delivery, via rebreathing or non-rebreathing circuit, of oxygen (1.5-2.5 L) and 1.0-2.5% isoflurane for anesthesia maintenance. Zofran 1.0 mg/kg (subcutaneous, SC), and dexamethasone 0.5 mg/kg (IM), was given immediately after intubation to control for nausea and inflammation, respectively. Hydration was maintained with lactated Ringer's solution (LRS) plus 5% dextrose at 4-10 mL/kg/h (i.v.). Isoflurane and fluid levels were adjusted during the course of a study to maintain an anesthesia.

Body temperature was maintained at 35-40° C. using a heated water blanket. Vital signs, including heart rate, blood pressure, respiration rate, oxygen saturation, and body temperature, were monitored at least every 10 to 15 min throughout the period in which the monkey was under anesthesia. Glycopyrrolate (0.01 mg/kg IM) was administered at the end of the study to control intubation-induced sialorrhea.

### Image Acquisition and Processing

Dynamic data were acquired in the MicroPET Focus-220 camera (Siemens Microsystems, Knoxville, TN). Emission data were collected for 120 min post-injection and reconstructed into a series of 33 frames and all standard corrections were applied: normalization, random, scatter and attenuation. Dynamic brain PET images were transferred and analyzed using the image processing PMOD software package v3.802 (PMOD Technologies, Zurich, Switzerland). The PET images were rigidly aligned to a previously acquired animal's brain T1 MRI and then spatially normalized to a common cynomolgus MRI template for anatomical brain region definition.

### Analysis Methods

### Assessing NBI-750142 Concentration

During each scan, whole blood PK samples were collected (in K2 EDTA tubes) at pre-dose (−5 min), and post-dose at 1 (end of bolus), 30, 60 (just prior to [¹⁸F]AV-133 radiopharmaceutical administration), 90, 120, 150, 180 (end of scan) minutes after test article administration. PK samples were approximately 1 mL each (8 mL total, including pre-dose sample). PK sample collection times were relative to start of NBI-750142 bolus administration.

NBI-750142 concentration in cynomolgus monkey plasma samples was analyzed using a qualified LC-MS/MS bioanalytical method. The assay had a quantifiable range from 0.25 to 250 ng/mL and with a dilution integrity demonstrated up to 1250 ng/mL. Plasma samples were processed using protein precipitation extraction with 0.1% formic acid in acetonitrile and the addition of stable deuterium isotope labeled internal standard NBI-750142-D6. Processed samples were chromatographically separated on a C18 column and coupled to a Sciex API 4000 triple quadrupole mass spectrometer. NBI-750142 concentrations are presented in Table 2-6.

### Determination of NBI-750142 in Cynomolgus Monkey K2-EDTA Plasma by LC-MS-MS

The method used in this study was qualified for a range of 0.250 to 250 ng/mL based on the analysis of 100 μL of plasma by LC-MS-MS. Quantitation was performed using a weighted 1/×2 linear least squares regression analysis generated from calibration standards. The protein precipitation extraction procedure began with the addition of internal standard solution (20.0 μL of approximately 100 ng/mL deuterated NBI-750142-D6). After adding 400 μL of acetonitrile/formic acid (1000:1.00), the tubes were sealed, vortexed and centrifuged. Subsequently, 100 μL of the supernatant was transferred to a new plate containing 400 μL of water/formic acid (1000:1.00). Chromatographic separation was achieved with a HPLC Kinetex C18 column (2.6 μm, 2.1×50 mm) and gradient elution using mobile phase A (water/formic acid; 1000:1.00) and mobile phase B (acetonitrile/methanol/formic acid; 500:500:1.00). A triple quadrupole mass spectrometer (Sciex API 4000) equipped with TurboIonSpray source was used in the positive ion mode. Quantitation was based on multiple reaction monitoring (MRM) of m/z transitions of NBI-750142 and NBI-750142-D6.

### Visualizing Tissue Uptake

Volumes of interest (VOIs) defined in the cynomolgus template space (see e.g., Ballanger et al. Neuroimage, 2013, 7:26-43) were applied to the spatially normalized PET images to compute time-activity curves (TACs, kBq/cm³) for caudate (0.96 cm³), putamen (1.34 cm³) and cerebellum (3.81 cm³). TACs and images (averaged between 60-120 min) were presented in Standard Uptake Value (SUV) units (g/mL) normalized by the weight of the animal and the injected dose.

### Image Quantification

Non-invasive Logan graphical analysis (start time of fit, t*=35 min) was used to estimate regional non-displaceable binding potential (BPND) using the TACs in the caudate and putamen, with the cerebellar cortex as the reference TAC. BPND was calculated in the in the caudate and putamen across baseline and NBI-750142 scans. Target occupancy was calculated according to Equation 1-1.

$$Occ ( \% ) = ( {BP}_{ND}^{Baseline} - {BP}_{ND}^{NBI - 750142} ) / {BP}_{ND}^{Baseline} \times 100. \qquad Equation 1 - 1$$

The relationship between the occupancy at VMAT2, averaged between caudate and putamen (i.e., striatum), and the administered dose or the average plasma concentration of NBI-750142 during the time of the scan was determined with a single specific binding site Eₘₐₓ model with a fixed maximum occupancy of 100%, according to Equation 1-2.

$$Occ = \frac{100 \% \times {C}^{h}}{{EC}_{50}^{h} + {C}^{h}} . \qquad Equation 1 - 2$$

where h is the Hill slope (h=1.0) and C is the mean of observed plasma concentrations during the scanning interval (60 to 180 minutes post-dose).

### Results

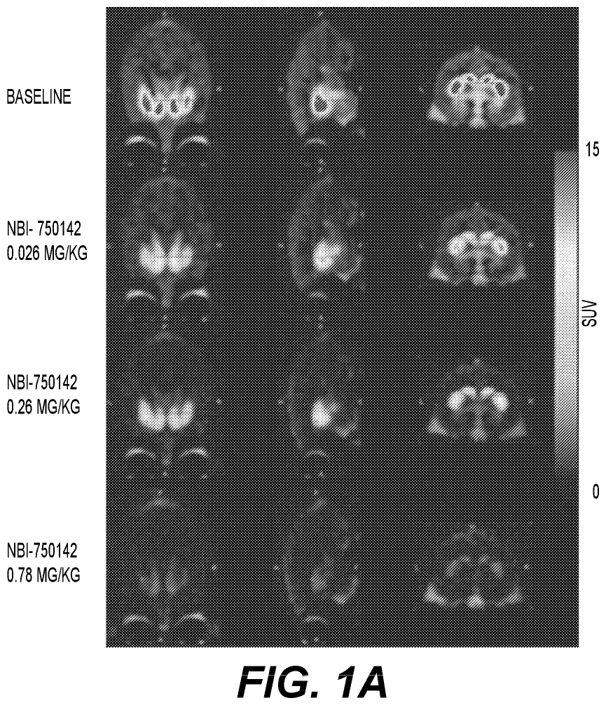

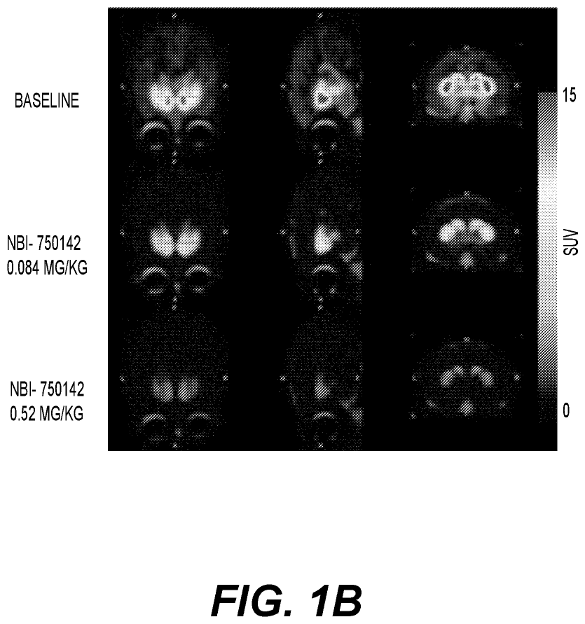

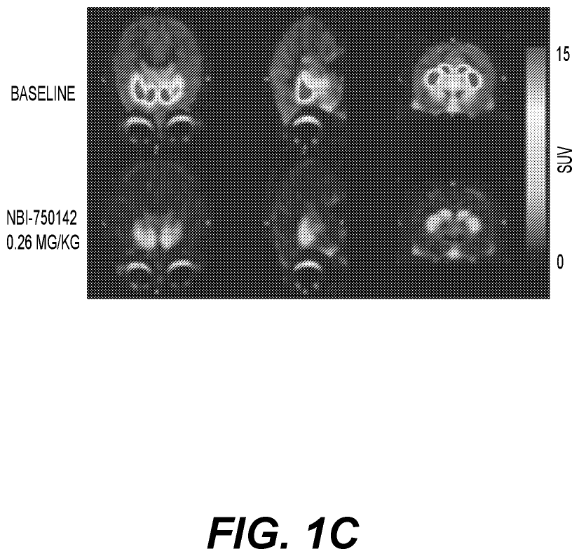

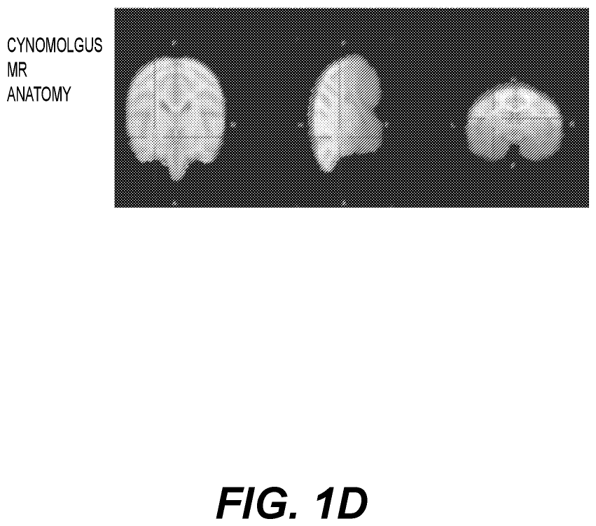

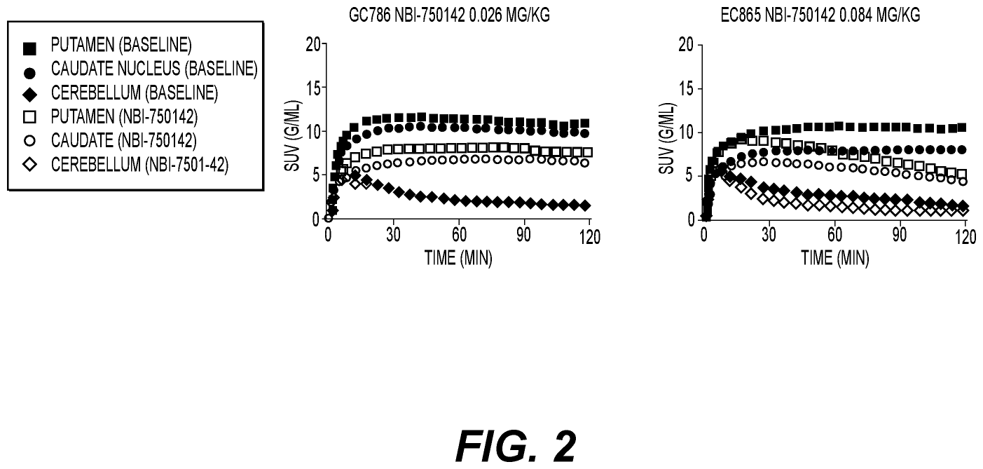

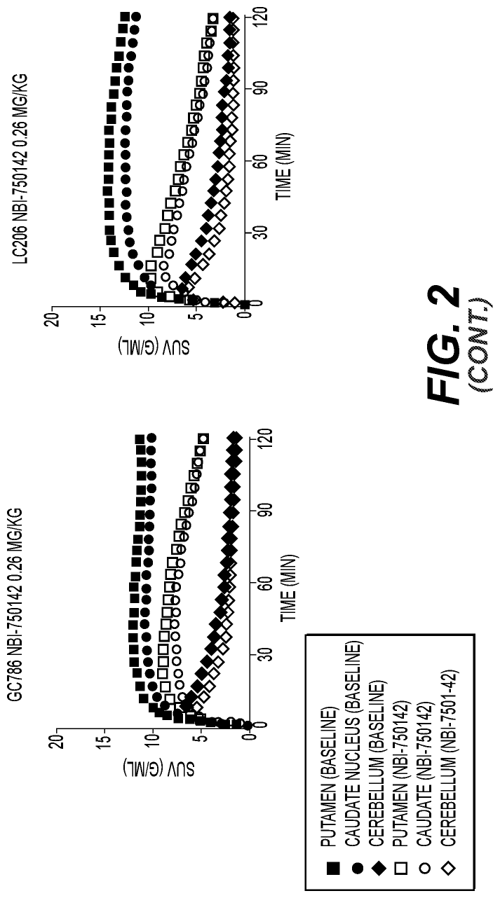

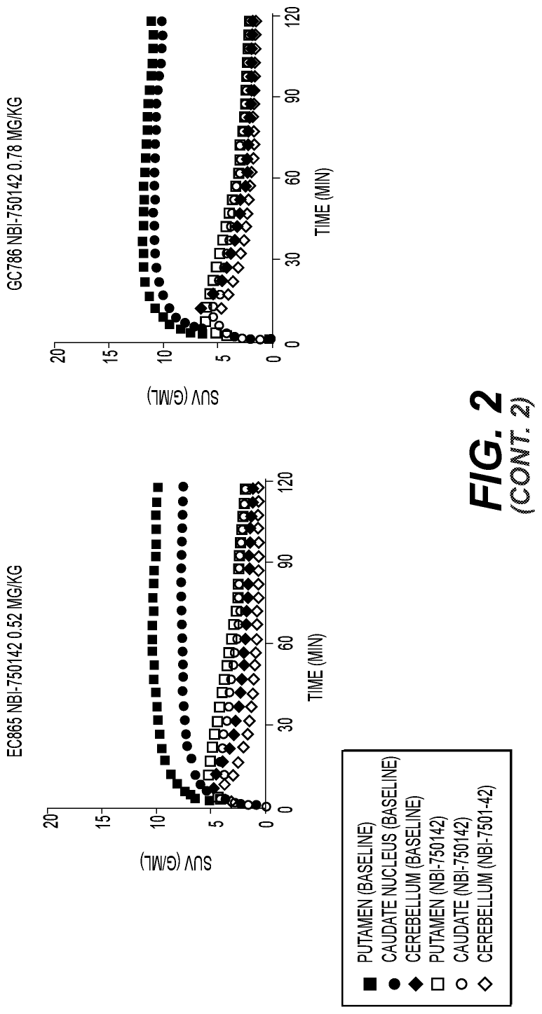

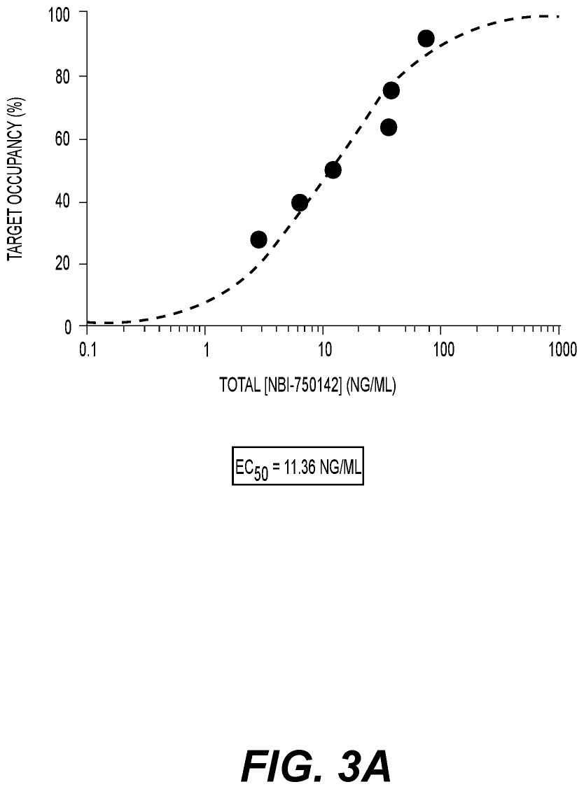

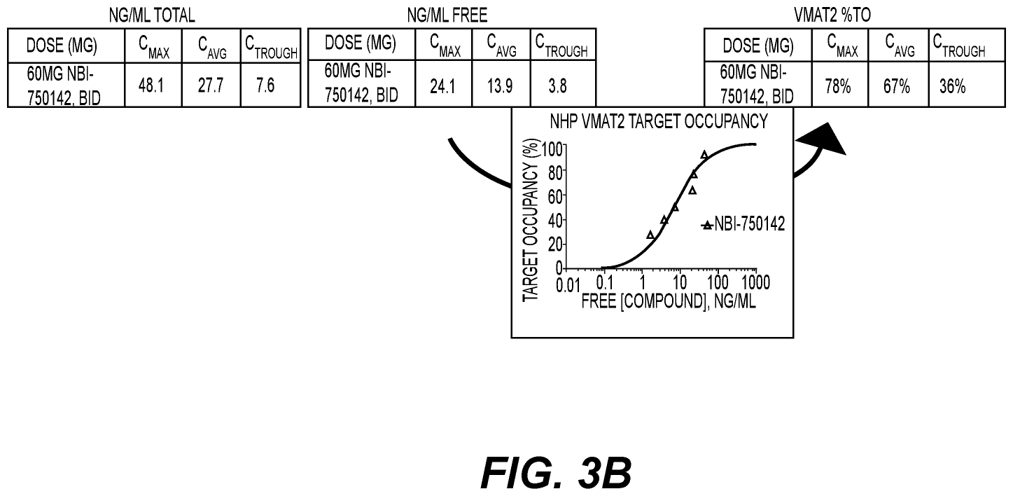

Three cynomolgus monkeys were imaged with [¹⁸F]AV-133 (3.33±0.45 mCi) at baseline and after administration of NBI-751042 to examine the relationship of average total NBI-750142 concentration during the time of the scan (mean concentration from 60-180 minutes post-NBI-750142 dose) and occupancy at VMAT2 in the striatum (occupancy averaged from caudate and putamen). Table 2-2 shows [¹⁸F]AV-133 and NBI-750142 injection information for each evaluation. Across evaluations, [¹⁸F]AV-133 SUV images are shown in FIGS. 1A-1D and TACs are shown in FIG. 2. At baseline, there was high uptake of [¹⁸F]AV-133 in the caudate and putamen relative to the rest of the brain. A dose-dependent decrease in [¹⁸F]AV-133 binding was observed post administration of NBI-750142. BPND and occupancy are provided in Table 2-3, Table 2-4 and Table 2-5 for evaluations in animals LC206, EC865 and GC786, respectively. Total NBI-750142 concentration and [¹⁸F]AV-133 striatal occupancy are provided in Table 2-6. The relationship of occupancy of NBI-750142 with total plasma concentration is shown in FIG. 3A, where the 50% effective total concentration (EC₅₀) was 11 ng/mL.

TABLE 2-3
[¹⁸F]AV-133 BP_{ND} and occupancy (% Occ) in animal LC206

| | | NBI-750142 | % Occ |
|---|---|---|---|
| Region | Baseline | 0.26 mg/kg | 0.26 mg/kg |
| Caudate | 4.00 | 1.42 | 64.5 |
| Putamen | 4.43 | 1.61 | 63.7 |

TABLE 2-4
[¹⁸F]AV-133 BP_{ND} and occupancy (% Occ) in animal EC865

| | | NBI- | NBI- | | |
|---|---|---|---|---|---|
| | | 750142 | 750142 | % Occ | % Occ |
| | | 0.084 | 0.52 | 0.084 | 0.52 |
| Region | Baseline | mg/kg | mg/kg | mg/kg | mg/kg |
| Caudate | 3.90 | 2.39 | 1.01 | 38.7 | 74.2 |
| Putamen | 5.24 | 3.10 | 1.18 | 40.9 | 77.5 |

TABLE 2-5
[¹⁸F]AV-133 BPND and occupancy (% Occ) in animal GC786

| | | NBI- | NBI- | NBI- | | | |
|---|---|---|---|---|---|---|---|
| | | 750142 | 750142 | 750142 | % Occ | % Occ | % Occ |
| | | 0.026 | 0.26 | 0.78 | 0.026 | 0.26 | 0.78 |
| Region | Baseline | mg/kg | mg/kg | mg/kg | mg/kg | mg/kg | mg/kg |
| Caudate | 4.50 | 3.10 | 2.22 | 0.33 | 31.1 | 50.8 | 92.7 |
| Putamen | 4.76 | 3.53 | 2.39 | 0.43 | 25.8 | 49.7 | 90.9 |

TABLE 2-6
NBI-750142 Measurements, Mean NBI-750142 Concentration Throughout
PET Scan, and Target Occupancy during PET Scan

| Animal | LC206 | GC786 | | EC865 | | |
|---|---|---|---|---|---|---|
| Dose Date | Apr. 1, 2019 | Jun. 17, 2019 | Apr. 22, 2019 | May 6, 2019 | Jun. 3, 2019 | Jun. 17, 2019 |
| Total Mass | 0.26 mg/kg | 0.026 mg/kg | 0.26 mg/kg | 0.78 mg/kg | 0.084 mg/kg | 0.52 mg/kg |
| Dose | | | | | | |
| ng/mL NBI- | BLQ* | BLQ | BLQ | BLQ | BLQ | BLQ |
| 750142, | | | | | | |
| Predose | | | | | | |
| ng/mL NBI- | 119 | BLQ | 102 | 186 | 108 | 295 |
| 750142 at | | | | | | |
| 1 min | | | | | | |
| ng/mL NBI- | 31.0 | 2.31 | 18.4 | 63.0 | 7.42 | 45.1 |
| 750142 at | | | | | | |
| 30 min | | | | | | |
| ng/mL NBI- | 26.0 | 2.24 | 10.6 | 65.2 | 6.04 | 36.9 |
| 750142 at | | | | | | |
| 60 min | | | | | | |
| ng/mL NBI- | 38.3 | 2.56 | 6.85 | 69.9 | 5.91 | 35.3 |
| 750142 at | | | | | | |
| 90 min | | | | | | |
| ng/mL NBI- | 41.1 | 2.69 | 13.4 | 75.1 | 5.85 | 36.2 |
| 750142 at | | | | | | |
| 120 min | | | | | | |
| ng/mL NBI- | 37.7 | 3.69 | 15.2 | 76.0 | 7.34 | 38.4 |
| 750142 at | | | | | | |
| 150 min | | | | | | |
| ng/mL NBI- | 35.2 | 3.10 | 15.3 | 86.5 | 7.07 | 41.3 |
| 750142 at | | | | | | |
| 180 min | | | | | | |
| Mean ng/mL | 35.7 | 2.9 | 12.3 | 74.5 | 6.4 | 37.6 |
| NBI-750142 | | | | | | |
| Concentration | | | | | | |
| during PET | | | | | | |
| Scan (60-180 | | | | | | |
| min) | | | | | | |
| Target | 64% | 28% | 50% | 92% | 40% | 76% |
| Occupancy, | | | | | | |
| Mean of | | | | | | |
| Putamen and | | | | | | |
| Caudate | | | | | | |

*BLQ = <0.250 ng/mL

### Conclusions

Concentration-dependent occupancy of NBI-750142 at VMAT2 was measured in NHP brain with [¹⁸F]AV-133 and PET. Using an Eₘₐₓ model with a Hill slope of 1, occupancy was related to total plasma NBI-750142 concentration with an EC₅₀ of 11 ng/mL. Following the same process used for NBI-98782 (see Example 1), the PET methods described above were applied to human pharmacokinetic data to estimate VMAT2 target occupancy (% TO) for NBI-750142 (see FIG. 3B). It was determined that 60 mg BID NBI-750142 (a dose expected to be the maximum allowable dose in humans) is estimated to achieve lower % TO than a moderately efficacious dose of valbenazine. Therefore, NBI-750142, as its maximum allowable dose, would not be expected to be as efficacious as valbenazine in the treatment of TD. These data serve as an example in which a VMAT2 compound can be assessed for further development at an early stage, using the process described herein, enabling effective and efficient decision-making for clinical development of VMAT2 inhibitors. Without being bound by theory, it is believed that the processes described in the Examples (e.g., matching cynomolgus monkey PET with human PK data) can be useful at least for developing tetrabenazine-analog VMAT2 inhibitors.

### Example 3. Open-Label, Positron Emission Tomography Study in Healthy Adult Male Subjects to Investigate VMAT2 Target Occupancy of Single Oral Doses of NBI-750142 Using [¹⁸F]AV-133

This study was conducted to evaluate the brain penetration and target engagement of VMAT2 after single oral doses of NBI-750142 in healthy subjects. This PET study assessed the engagement of NBI-750142 with VMAT2 in vivo at two time-points after dosing. A sequential-cohort design allowed for the safety, target-occupancy, and available pharmacokinetic (PK) data from each prior cohort to be evaluated before selection of the dose level for the next cohort. A range of NBI-750142 doses (max. 200 mg) were studied to explore the relationship between NBI-750142 plasma concentrations and engagement of VMAT2 in selected brain regions.

This was a Phase 1 single-center, open-label, single-dose, target occupancy study of NBI-750142 in up to 12 healthy male subjects using [¹⁸F]AV-133 PET imaging. The study had a sequential cohort design with up to 6 cohorts, where a single oral dose of NBI-750142 was administered to 2 to 4 subjects per dose cohort. A total of 12 subjects completed the study. Subjects enrolled in the study were required to report to the clinical research site for 4 separate occasions, detailed below.

### Screening Period (Days −28 to −2)

Dosing day is Day 1. Day−1 is the day before dosing day, Day−2 is 48 hours before dosing day, Day-n is n days before dosing day. Screening assessments were conducted to determine eligibility (within 28 days prior to administration of the study medication). All screening safety assessments were performed to ensure subjects are medically healthy. A brain magnetic resonance imaging (MRI) was acquired in all subjects to assess eligibility. The MRI was also utilized for anatomical localization and ROI (region of interest) analysis by co-registration to the subject's PET summation image.

Re-screening was allowable on study if a subject was deemed eligible but did not enroll within the 28-day screening window. During the re-screening visit, subjects repeated all screening safety assessments per protocol to ensure they were medically healthy with no newly incurred clinically significant medical history or clinically significant findings on physical examination, laboratory profiles, vital signs, or ECGs. As the brain MRI was not part of the panel of safety assessments, the MRI was not required to be repeated if performed within 1 year of the rescreening visit.

After completion of the screening assessments, eligible subjects were instructed to:
Refrain from taking prohibited medications
Refrain from consuming alcohol or caffeinated products for 48 hours before Day −1 (measured from dosing day)
Return to the study center for baseline assessments between Day −14 to Day −1 (measured from dosing day)

### Baseline Assessments (Day −14 to Day−1)

Subjects underwent baseline assessment procedures within 14 days before Day 1 (dosing day). On the day prior to the Baseline Visit, subjects were admitted to the study center. Subjects were medically assessed to ensure they met inclusion/exclusion criteria of the study. A urinary drug screen (UDS), urine cotinine, and urine alcohol test were performed on admission. The following day the baseline [¹⁸F]AV-133 PET scan was performed. If NBI-750142 dosing occurred on the day after the Baseline Visit, subjects were not discharged between baseline assessments and NBI-750142 dosing.

### Day Prior to Dosing (Day −1)

Subjects were admitted to the study center the day before NBI-750142 dosing (Day −1), if not already admitted and were discharged on Day 3.

### Treatment and Follow-Up Period (Day 1 to Day 3)

Two subjects were initially enrolled in each dose cohort. Each subject received one brain structural magnetic resonance imaging (MRI) scan during screening and up to 3 [¹⁸F]AV-133 PET scans (1 baseline and up to 2 follow-up scans after receiving a single dose of NBI-750142). Subjects enrolled in the first cohort received a single dose of 100 mg of NBI-750142 orally on Day 1. PET scans occurred on Day 1 at approximately 1.5 hours post NBI-750142 dose and on Day 2 at approximately 18 hours post NBI-750142 dose. Subjects were then discharged from the study center after all Day 3 procedures and assessments were completed.

### Final Study Visit (Day 10±2 or Early Termination)

Subjects returned to the center for a final study visit on Day 10 (±2 days). Safety assessments were performed including a physical and neurological examination, adverse event assessment, ECG, vital signs, and laboratory examinations.

### Inclusion & Exclusion Criteria

Subjects were required to meet the following inclusion criteria to be eligible for the study:
1. Male, aged 18 to 55 years, inclusive, at screening.
2. Be medically healthy with no clinically significant medical history or clinically significant findings on physical examination, laboratory profiles, vital signs, or ECG at screening or Day −1.
3. Have a body mass index (BMI) of ≥18 and ≤30 kg/m².
4. Male subjects and their partners of childbearing potential must commit to the use of two methods of contraception, one of which is a barrier method for the male subjects for the study duration.
5. Male subjects must not donate sperm for the study duration and for 90 days after study completion.
6. Be able to read, comprehend, sign, and date the informed consent form (ICF).
7. Be willing to comply with all study procedures and restrictions, including abstinence from strenuous, unaccustomed exercise and sports.
8. Agree to remain exclusively in the study unit for mandatory periods and return for all follow-up visits.

Subjects were required to not meet any of the following exclusion criteria to be eligible for the study:
1. Have an unstable psychological disorder ≤1 year before screening or have a significant risk of suicidal or violent behavior.
2. Have a history of seizures, epilepsy, significant brain injury or lesions, strokes or transient ischemic attacks, or prior intracranial or brain surgery.
3. Have current clinically significant cardiovascular disease or abnormalities on screening ECG, including but not limited to an average triplicate corrected QT interval using Fridericia's formula (QTcF) >450 msec at screening or baseline, or history of prolonged QT syndrome.
4. Have a positive human immunodeficiency virus antibody (HIV-Ab) test result, hepatitis B surface antigen (HBsAg) test result, or positive hepatitis C virus antibody (HCV-Ab) test result with a positive HCV-Ab polymerase chain reaction (PCR) result at screening or have a history of a positive result.
5. Have a hemoglobin value of <12 g/dL at screening.
6. Have aspartate aminotransferase (AST), alanine aminotransferase (ALT), gamma-glutamyl transferase (GGT), or total bilirubin levels greater than the upper limit of normal (ULN) at screening. Subjects with a documented diagnosis of Gilbert's syndrome are not required to meet the bilirubin criteria.
7. Have a positive alcohol urine test or UDS (positive for amphetamines, barbiturates, benzodiazepine, phencyclidine, cocaine, opiates, or cannabinoids) at screening or Day −1.
8. Consume more than 2 alcoholic beverages daily or more than 14 alcoholic beverages weekly ≤7 days of Day −1 or consume any alcohol within 48 hours of Day −1.
9. Used nicotine or cannabis products 60 days before Day −1.
10. Used prescription or over-the-counter (OTC) medications, with the exception of acetaminophen (within recommended dosing), 7 days before Day −1.
11. Used alternative/complementary medicinal products (e.g., herbal supplements, medicinal teas, creatine, sports supplements), with the exception of vitamins and minerals (excluding those supplemented with herbal preparations) 7 days before Day −1. Vitamins and minerals must be within the daily recommended dietary allowance (RDA) doses (e.g., a daily multivitamin).
12. Have taken a strong inducer of CYP3A4/5 (e.g., rifampin, carbamazepine, phenytoin, phenobarbital, rifabutin, primidone, St. John's Wort) ≤30 days before Day −1.
13. Have taken a strong inhibitor of CYP3A4/5 (e.g., ketoconazole, itraconazole, erythromycin, clarithromycin, ritonavir) within 14 days or 5 half-lives (whichever is longer) before Day −1.
14. Have taken a CYP2D6 inhibitor (e.g., bupropion, fluoxetine, paroxetine, quinidine) within 14 days or 5 half-lives (whichever is longer) before Day −1.
15. Have taken a monoamine oxidase inhibitor (MAOI) (e.g., isocarboxazid, tranylcypromide, phenelzine, selegiline, rasagiline) ≤30 days of Day −1.
16. Have taken a VMAT2 inhibitor (e.g., valbenazine, tetrabenazine, deutetrabenazine, or reserpine) 30 days before Day −1; [¹⁸F]AV-133 injection or NBI-750142 at any time.
17. Have taken any of the medications listed in Concomitant Treatment section that might interfere with [¹⁸F]AV-133 PET imaging 30 days before the baseline [¹⁸F]AV-133 injection: methylphenidate, reserpine, amphetamine derivative, dextroamphetamine derivative, methylphenidate derivative, or bupropion.
18. Are currently taking medications that are known to cause QT-prolongation.
19. Any other medical or psychiatric condition or laboratory abnormality, which in the opinion of the investigator might preclude participation.
20. Have a history of allergic response to a VMAT2 inhibitor (e.g., valbenazine, tetrabenazine, deutetrabenazine, or reserpine).
21. Have current substance abuse (e.g., analgesics, tranquilizers, opioids, stimulants, moodaltering drugs), or have a known drug dependence.
22. Self-report consumption of more than 5 caffeine-containing beverages a day in the past 30 days or self-report consumption of any caffeine-containing product within 48 hours before Day −1.
23. Have ingested grapefruit juice or grapefruit products 7 days before Day −1.
24. Have received any investigational product within a time period equal to 5 half-lives of the product, if known, or a minimum of 60 days before Day −1.
25. Have a history of, or suspected, poor compliance in clinical research studies.
26. Have a blood loss of ≥500 mL or donated blood within 56 days of Day −1.
27. Have any of the following findings on the brain MRI: findings of infectious disease, space-occupying lesions, normal pressure hydrocephalus, or any other abnormalities associated with CNS disease.
28. Have implants, such as implanted cardiac pacemakers or defibrillators, insulin pumps, cochlear implants, metallic ocular foreign body, implanted neural stimulators, CNS aneurysm clips, or other medical implants that have not been certified for MRI, or a history of claustrophobia in MRI.
29. Prior participation in other research protocols or clinical care in the last year in addition to the radiation exposure expected from participation in this clinical study, such that radiation exposure exceeds the effective dose of 50 mSv, which would be above the acceptable annual limit established by the US Federal Guidelines.
30. Have been deemed unacceptable to participate by the study investigator.

### Concomitant Treatment

All prescription and OTC medications, dietary supplements (including vitamins), and herbal supplements taken by subjects during the 30 days before screening were recorded. The use of any prescription and OTC medications and alternative/complementary medicinal products (e.g., herbal supplements, medicinal teas, creatine, sports supplements), with the exception of vitamins and minerals (excluding those supplemented with herbal preparations), and acetaminophen (within recommended dosing), were prohibited within 7 days before Day −1 until the end of the study. Vitamins and minerals must have been within the daily RDA (recommended dietary allowance) doses (e.g., a daily multivitamin).

The following medications were prohibited in the study:
Strong inducer of CYP3A4/5 (e.g., rifampin, carbamazepine, phenytoin, phenobarbital, rifabutin, primidone, St. John's Wort)≤30 days before Day −1.
Strong inhibitor of CYP3A4/5 (eg, ketoconazole, itraconazole, erythromycin, clarithromycin, ritonavir) within 14 days or 5 half-lives (whichever is longer) before Day −1.
CYP2D6 inhibitor (e.g., bupropion, fluoxetine, paroxetine, quinidine) within 14 days or 5 half-lives (whichever is longer) before Day −1.
MAOI (e.g., isocarboxazid, tranylcypromide, phenelzine, selegiline, rasagiline)≤30 days of Day −1.
VMAT2 inhibitor (e.g., valbenazine, tetrabenazine, deutetrabenazine, or reserpine) 30 days before Day−1 and [18F]AV-133 injection or NBI-750142 at any time.
Prescription or OTC medications, except acetaminophen (within recommended dosing) ≤7 days before Day −1.
Alternative or complementary medicinal products (e.g., herbal supplements, medicinal teas, creatine and sports supplements), except vitamins and minerals (excluding those supplemented with herbal preparations) within the daily recommended daily allowance ≤7 days before Day −1.

The following list of medications may have an interference with [¹⁸F]AV-133 PET imaging and were not allowed within 30 days before the baseline [¹⁸F]AV-133 injection:
Methylphenidate
VMAT2 inhibitor
Amphetamine derivative
Dextroamphetamine derivative
Methylphenidate derivative
Bupropion

### Dietary and Other Restrictions

Subjects were required to fast during the study. Subjects were required to abstain from large meals at least 8 hours prior to NBI-750142 dosing. If fasting was more than 8 hours, subjects received a full breakfast (provided 8 hours prior to dosing). All subjects received a small predefined snack that was consumed at least 2 hours prior to NBI-750142 dosing. Subjects were not allowed to consume any food for at least 3 hours after NBI-750142 administration and were not allowed to consume food during PET scans.

Subjects were not allowed to consume water (other than that required for dosing, 240 mL) from 1 hour prior to 1 hour after NBI-750142 dosing. Water was allowed during all other fasting periods. Subjects were also required to fast prior to the baseline and second post-dose PET scan in the same way as the first post-dose PET scan.

Additional dietary restrictions included:
Alcohol
Caffeinated products
Nicotine or cannabis products
Grapefruit or grapefruit juice

### NBI-750142 Study Drug

NBI-750142 was constituted with water as a solution no more than 72 hours prior to dosing. The appropriate volume of dosing solution (based on the cohort) was administered by an oral dosing syringe. Following dosing, subjects were instructed to consume a predetermined volume of water so that the total administered volume of solution plus water equaled 240 mL. Subjects had to ingest the dosing solution and water within 2 minutes.

### [¹⁸F]AV-133 Imaging Procedures

Subjects were positioned in a gently securing head holder using the laser lights of the camera so that the brain is centered in the field of view. Prior to the radiotracer injection and emission imaging, a CT scan was performed to provide correction coefficients for photon attenuation due to matter.

Subjects were administered a single dose of [¹⁸F]AV-133 intravenously over 3 minutes using an infusion pump (approximately 3.33 mL per minute) through a venous catheter followed by a 10 mL saline flush. Dynamic PET imaging of the brain was acquired over 120 min following radiotracer injection on a Siemens Biograph 6 PET/CT camera according to the following protocol:
CT scan of the head
[¹⁸F]AV-133 injection (T=0 min)
Emission scan: 6×30 sec, 4×1 min, 4×2 min, 21×5 min (0-120 min)

Images were reconstructed in a 168×168×81 matrix (pixel size of 2.03 mm×2.03 mm) with an iterative reconstruction algorithm (OSEM 4 iterations, 16 subsets) and a post hoc Gaussian filter=3 mm. Standard corrections for random, scatter, system dead time and attenuation provided by the camera manufacturer were performed.

### MRI Brain Imaging

An MRI scan was acquired from eligible subjects as part of the screening visit, to identify and delineate brain anatomical regions of interest for individual PET images. MRI scans were obtained on a Siemens Espree 1.5 Tesla clinical magnet. MRI scans of 1 mm contiguous slices were obtained with the following sequence: Sagittal T1, axial T2 double echo, axial flair, coronal T2, axial DWI, axial T1, sagittal MPRAGE.

### [¹⁸F]AV-133 Image Analysis

Reconstructed PET imaging data volumes were transferred to the image processing PMOD software package (PMOD Technologies, Zurich, Switzerland) where the images were motion and decay corrected, realigned onto the subject MRI and subsequently normalized into the standard MNI (Montreal Neurological Institute) space where volumes of interest (VOIs) were defined from a template (see e.g., Hammers et al, Adult brain maximum probability map (“Hammersmith atlas”; n30r83) in MNI space. 2008). The subject MRI was segmented into grey matter, white matter, and CSF maps. Average activity concentration (kBq/cc) within each VOI, constrained to grey matter voxels for cortical regions, were determined and time activity curves (TACs) were generated. TACs were extracted from the following VOIs: caudate and putamen (striatum), midbrain, cerebellar and occipital cortices. TACs and were expressed in standard uptake value (SUV) units (g/mL) by normalizing with the weight of the subject and the injected dose. The occipital cortex was used as a reference region to normalize activity in target regions to generate SUVr images. For visualization, standard uptake value ratio (SUVr) images were computed between 60-120 min. Non-displaceable binding potential (BPND), the primary outcome measure, was estimated with noninvasive Logan graphical analysis (NI-LGA, t*=20 min) in caudate, putamen and midbrain using the occipital cortex as the reference region. Percent measured occupancy (OM) of NBI-750142 (drug) by region was computed according to the Equation 3-1 at each of the post-dose timepoints, T1 (1.5 h post dose) and T2 (18 h post dose).

$${O}^{M} = 1 \frac{{BP}_{ND} ( drug )}{{BP}_{ND} ( baseline )} \times 100. \qquad Equation 3 - 1$$

The Eₘₐₓ model, Equation 3-2, was used to describe the relationship between NBI-750142 plasma concentration (ng/mL, mean of scan start and scan end measurements) and occupancy in the striatum (mean occupancy of caudate and putamen) and midbrain where Eₘₐₓ was constrained to 100, i.e., maximum occupancy reaches 100% at a sufficiently high concentration, Equation 3-2.

$${O}^{M} = \frac{[ Drug ]}{[ Drug ] + {EC}_{50}} . \qquad Equation 3 - 2$$

where [Drug] is the concentration of NBI-750142 (ng/mL) measured in plasma and EC₅₀ is the estimated plasma drug concentration that would achieve 50% VMAT2 occupancy, as measured by displacement of [¹⁸F]AV-133.

Post-hoc exploratory analysis applied an Upregulation model (Equation 3-3), in order to account for the apparent ‘negative occupancy’ values observed at T2 (BPND values at T2 were unexpectedly higher than corresponding BPND values at baseline).

$${O}^{M} = \frac{[ Drug ] - {EC}_{50} \frac{[ Drug ]}{[ Drug ] + {EC}_{50}} ( URF - 1 )}{[ Drug ] + {EC}_{50}} . \qquad Equation 3 - 3$$

For the Upregulation model (Equation 3-3), an upregulation factor (URF) and EC₅₀ were estimated (and complete occupancy (100%) was assumed at sufficiently high drug concentration). The Upregulation factor, URF, in the model is neither time-dependent nor concentration dependent, i.e., upregulation is applied to all data points, regardless of when they were acquired.

### Study Results

### Subject Demographics

A total of 20 healthy male volunteers were screened to enroll a total of 12 participants. At the time of screening, the participants ages were 29-54 years (mean 43.2 years). Nine of the enrolled subjects were Black or African American, and three were Caucasian. One subject identified as Hispanic or Latino and eleven subjects identified as not Hispanic or Latino. Subject demographics are provided in Table 3-1.

TABLE 3-1
Subject Demographics

| | NBI- | NBI- | NBI- | NBI- | |
|---|---|---|---|---|---|
| | 750142 | 750142 | 750142 | 750142 | A11 |
| | 10 mg | 60 mg | 100 mg | 200 mg | Subjects |
| | (N = 4) | (N = 4) | (N = 2) | (N = 2) | (N = 12) |
| Age (years) | | | | | |
| n | 4 | 4 | 2 | 2 | 12 |
| Mean | 41.3 | 38.8 | 53.5 | 45.5 | 43.2 |
| SD | 9.0 | 10.8 | 0.7 | 4.9 | 9.2 |
| Median | 43.0 | 36.0 | 53.5 | 45.5 | 43.5 |
| Min, Max | 29, 50 | 29, 54 | 53, 54 | 42, 49 | 29, 54 |
| Sex, n (%) | | | | | |
| Male | 4 (100) | 4 (100) | 2 (100) | 2 (100) | 12 (100) |
| Ethnicity, n (%) | | | | | |
| Hispanic or | 1 (25.0) | 0 | 0 | 0 | 1 (8.3) |
| Latino | | | | | |
| Not Hispanic | 3 (75.0) | 4 (100) | 2 (100) | 2 (100) | 11 (91.7) |
| or Latino | | | | | |
| Race, n (%) | | | | | |
| American | 0 | 0 | 0 | 0 | 0 |
| Indian or | | | | | |
| Alaska | | | | | |
| Native | | | | | |
| Asian | 0 | 0 | 0 | 0 | 0 |
| Black or | 2 (50.0) | 4 (100) | 1 (50.0) | 2 (100) | 9 (75.0) |
| African | | | | | |
| American | | | | | |
| White | 2 (50.0) | 0 | 1 (50.0) | 0 | 3 (25.0) |
| Native | 0 | 0 | 0 | 0 | 0 |
| Hawaiian or | | | | | |
| Other Pacific | | | | | |
| Islander | | | | | |
| Other | 0 | 0 | 0 | 0 | 0 |
| Multiple | 0 | 0 | 0 | 0 | 0 |

*Percentages are based on the number of subjects in the safety analysis set (N).

### [¹⁸F]AV-133 Brain Images and Time Activity Curves

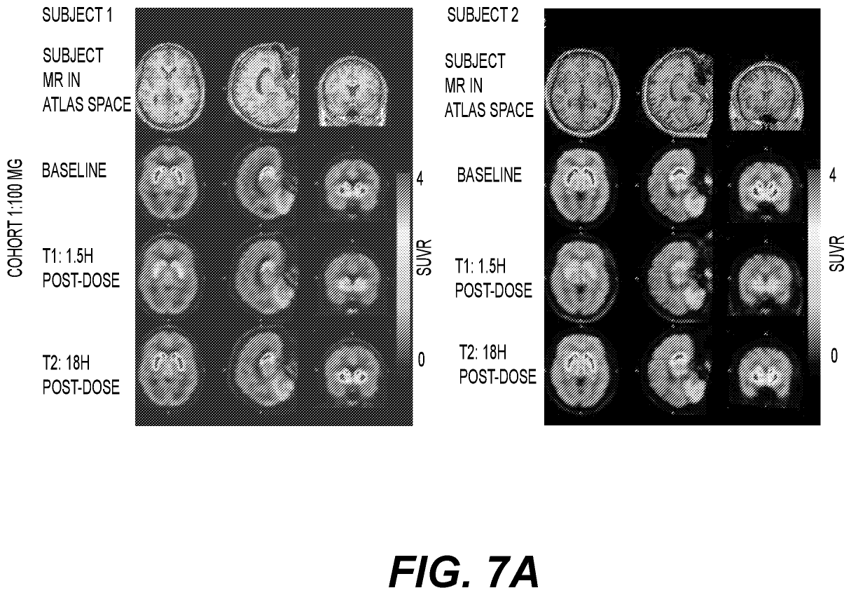

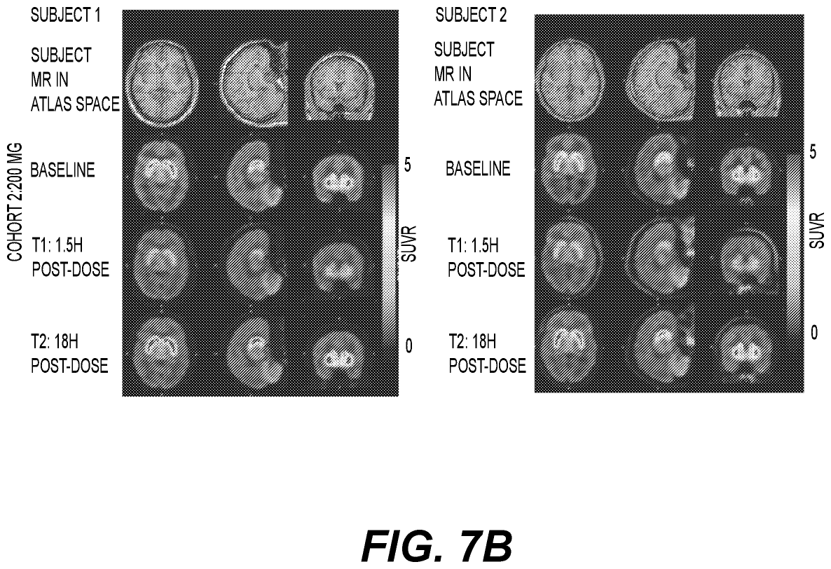

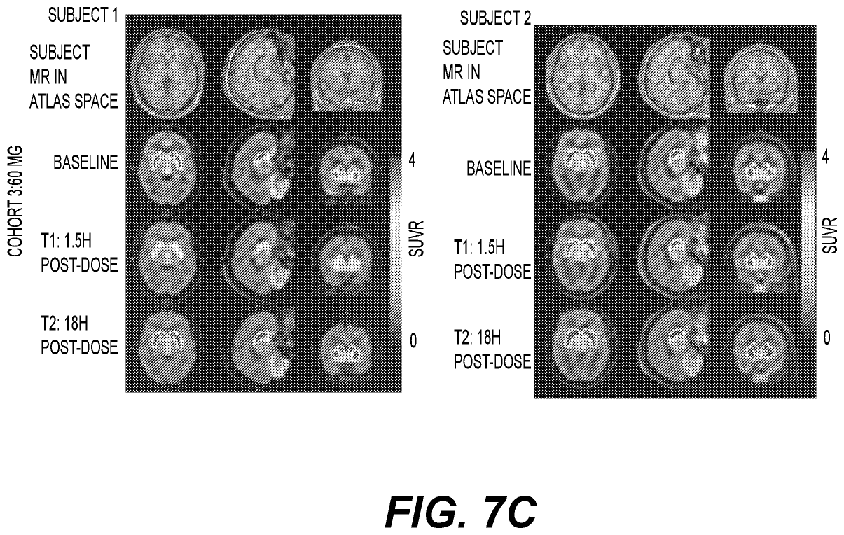

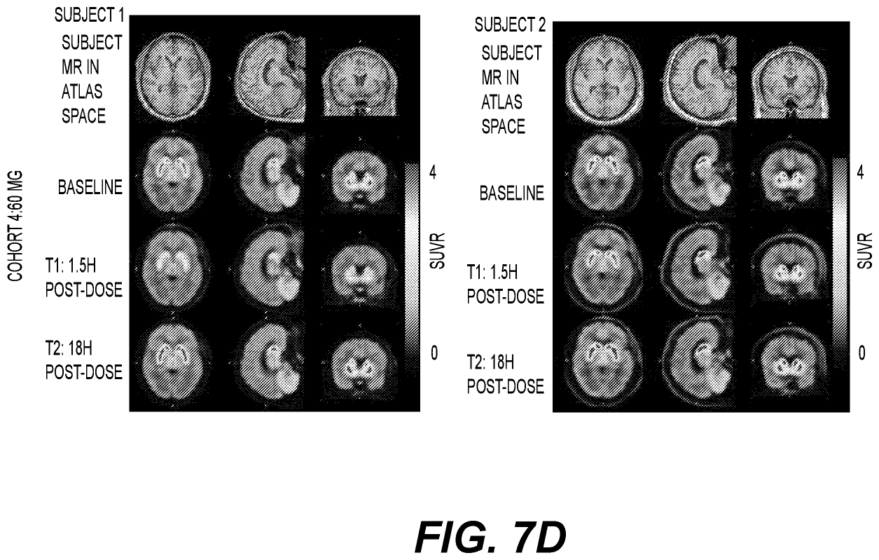

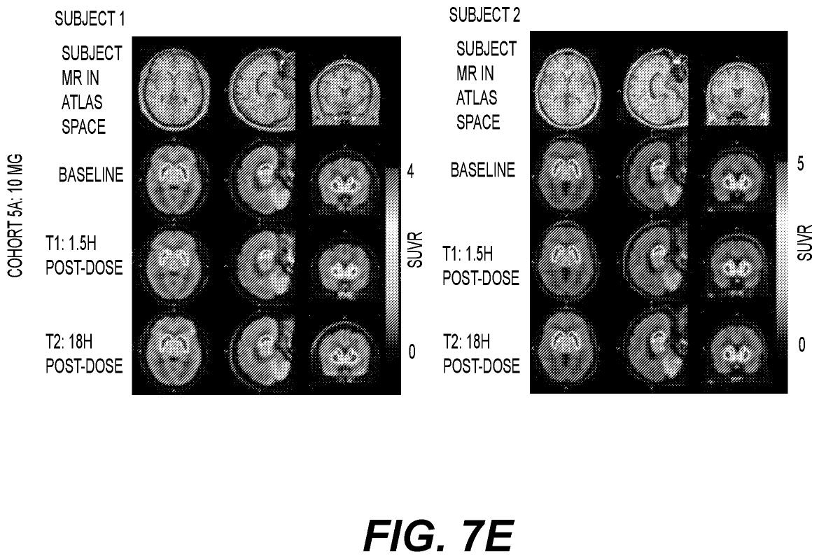

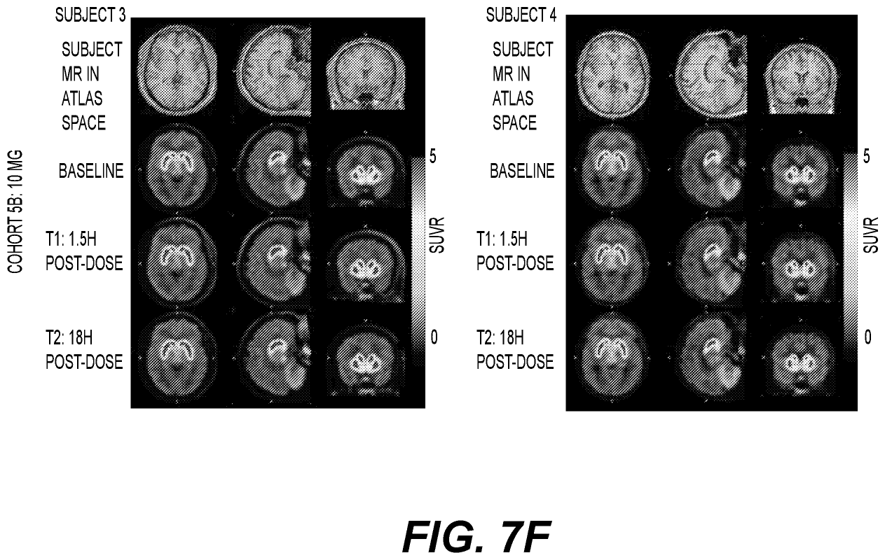

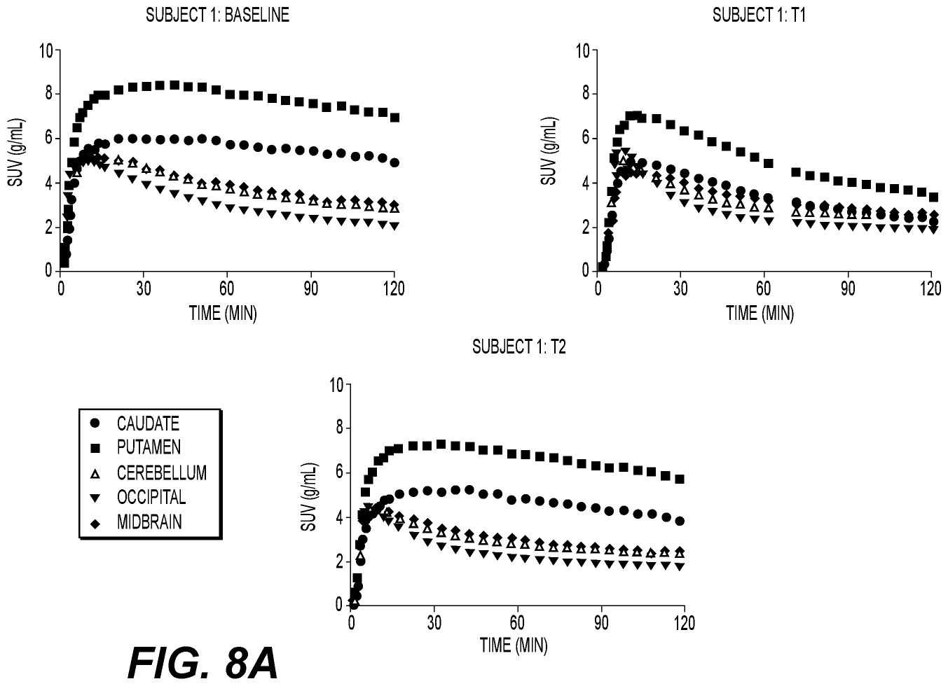

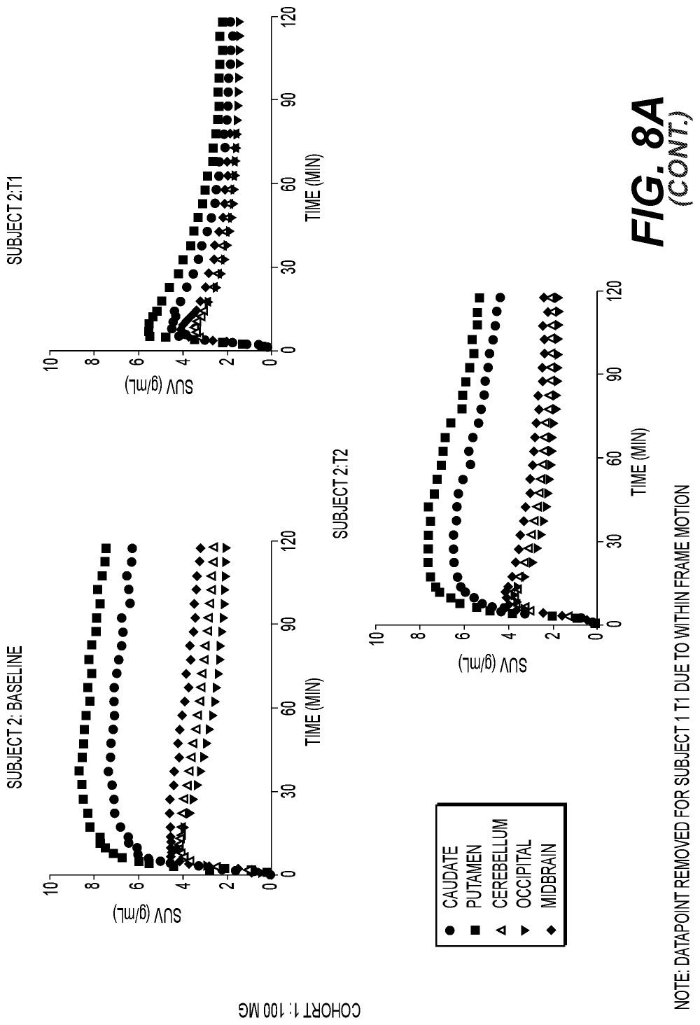

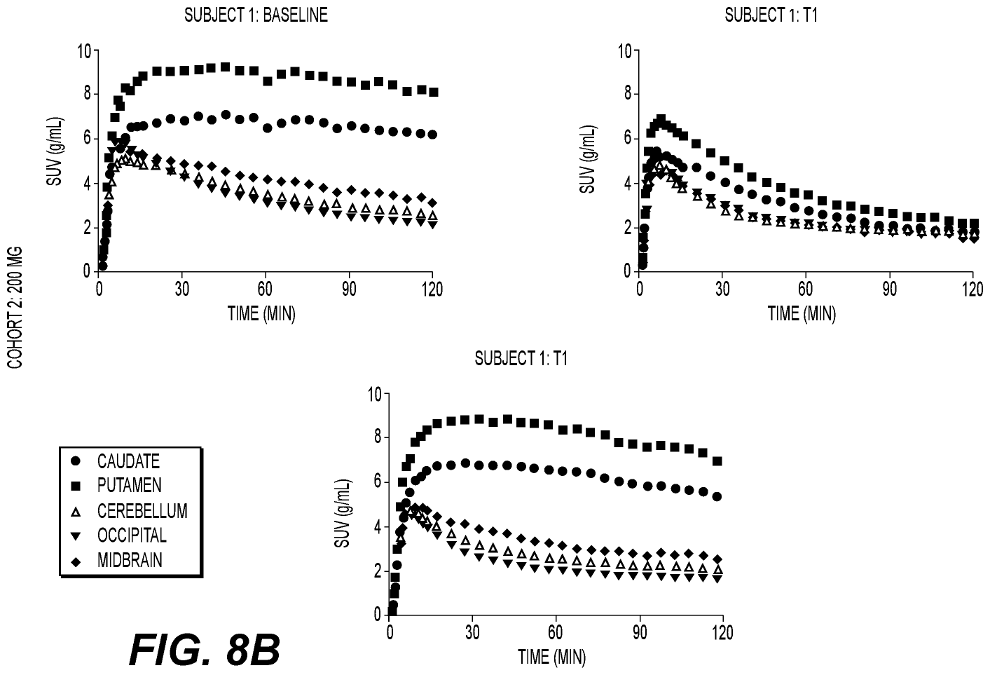

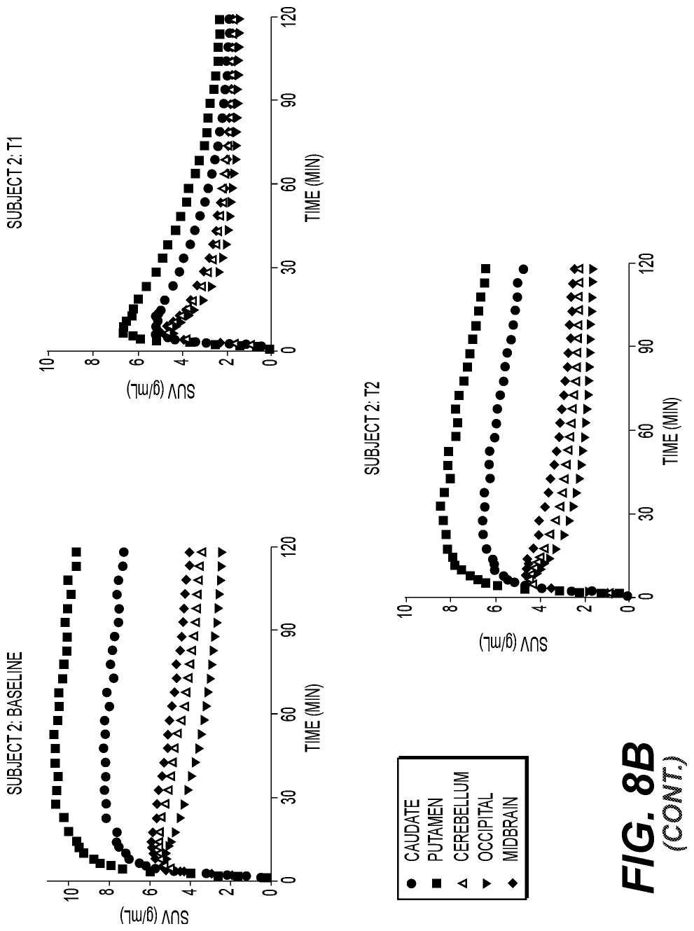

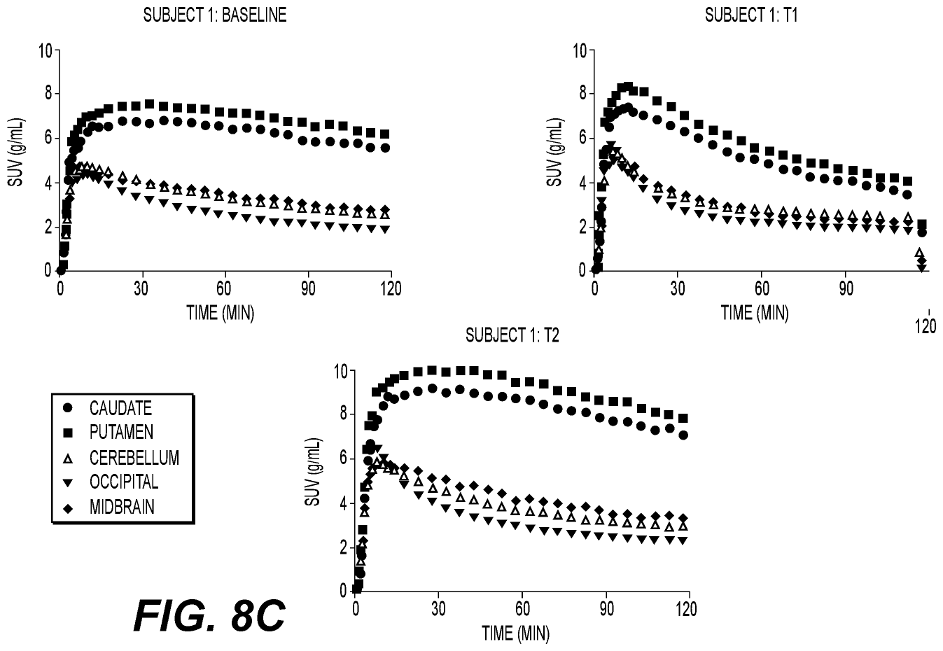

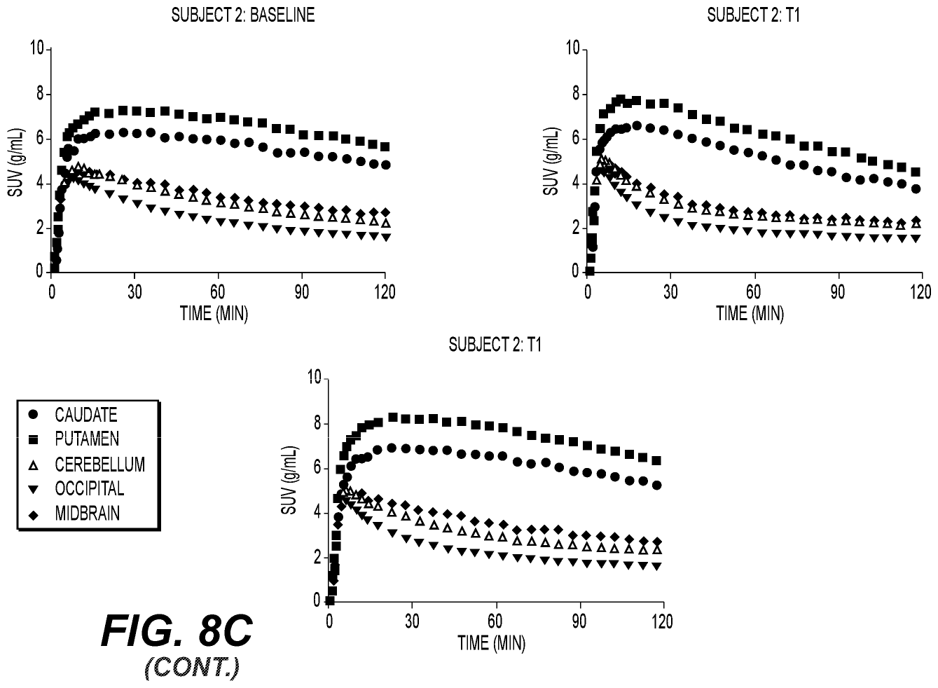

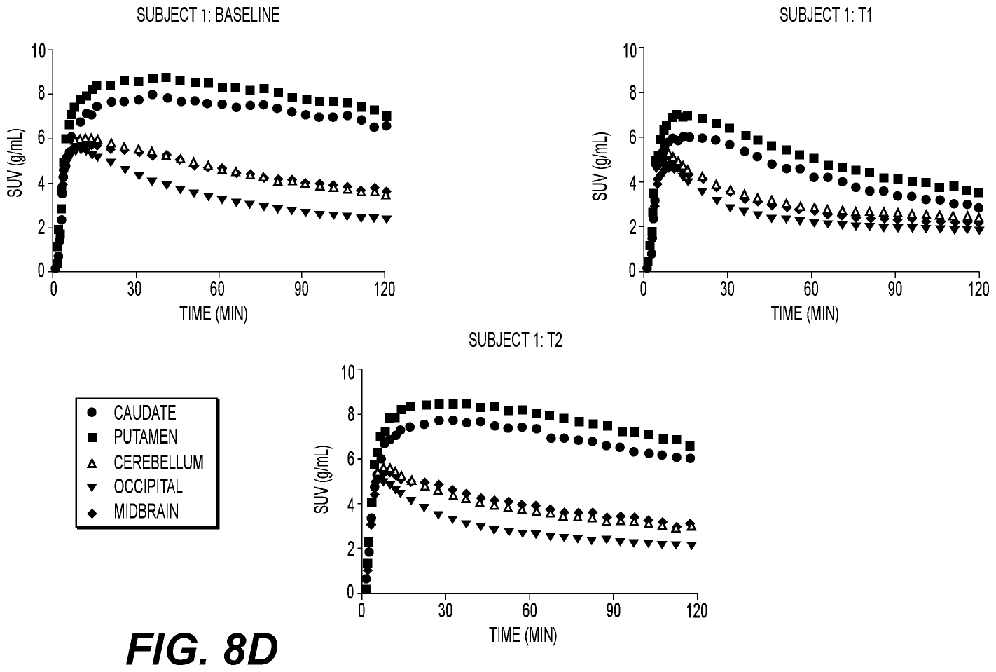

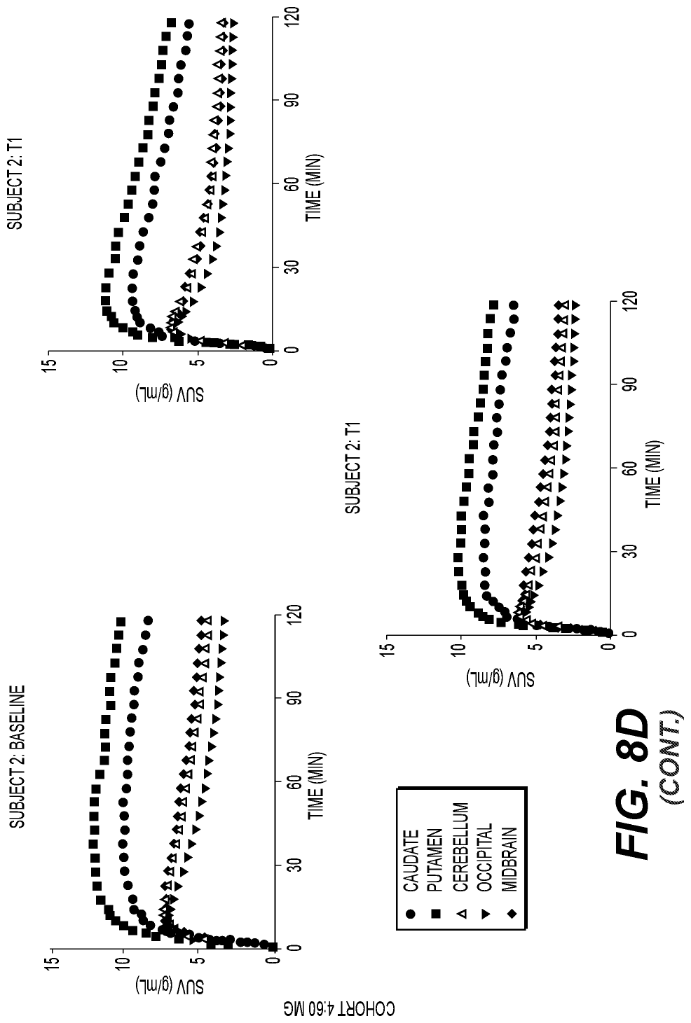

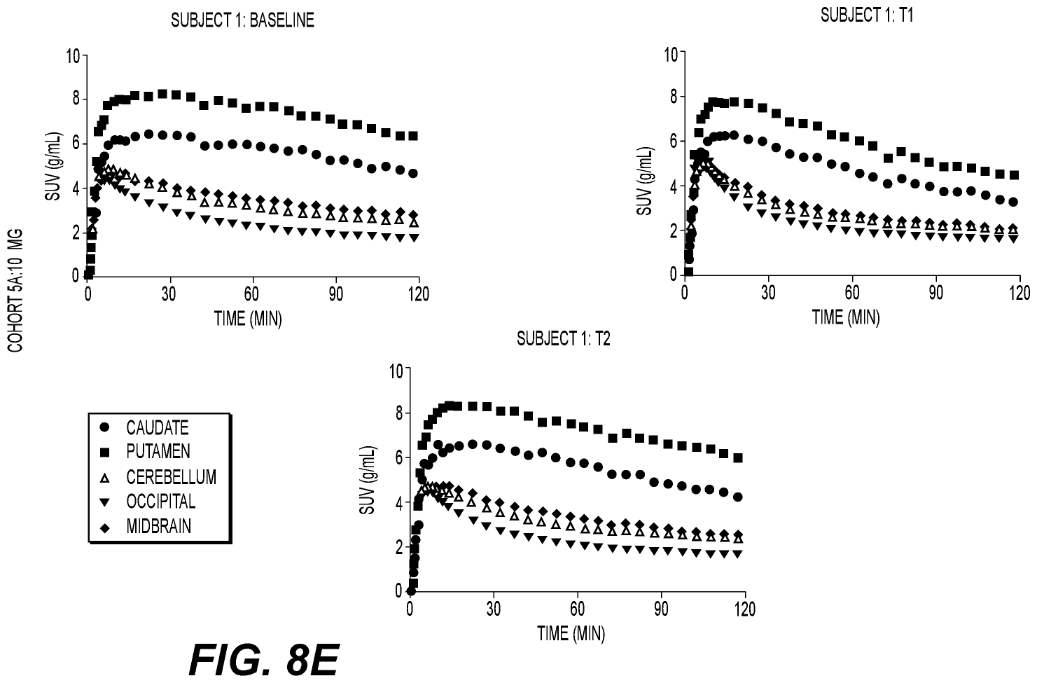

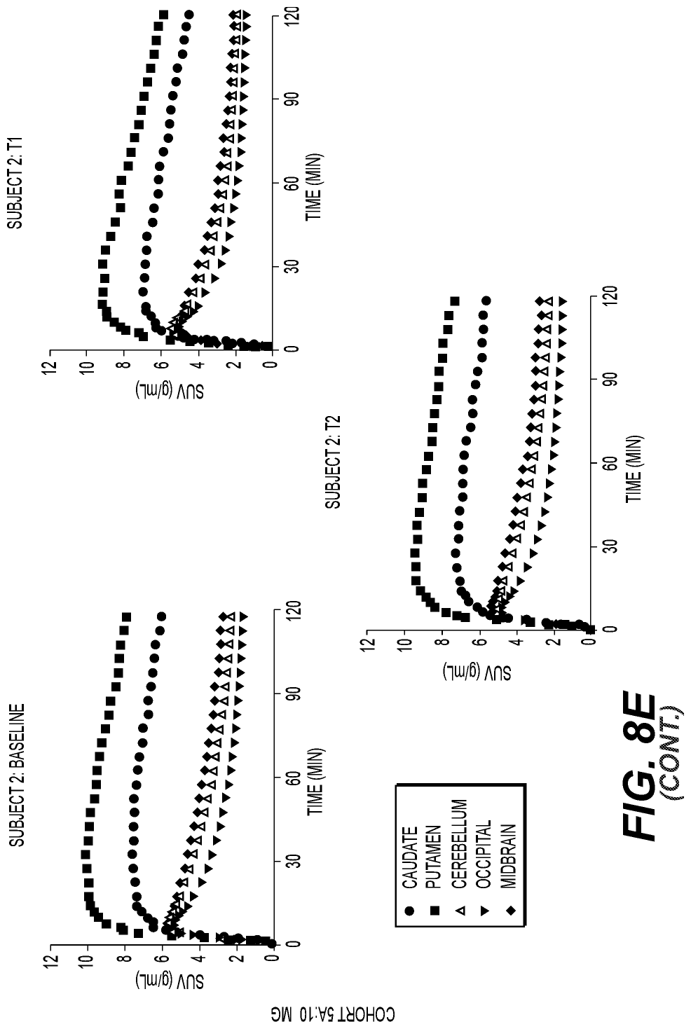

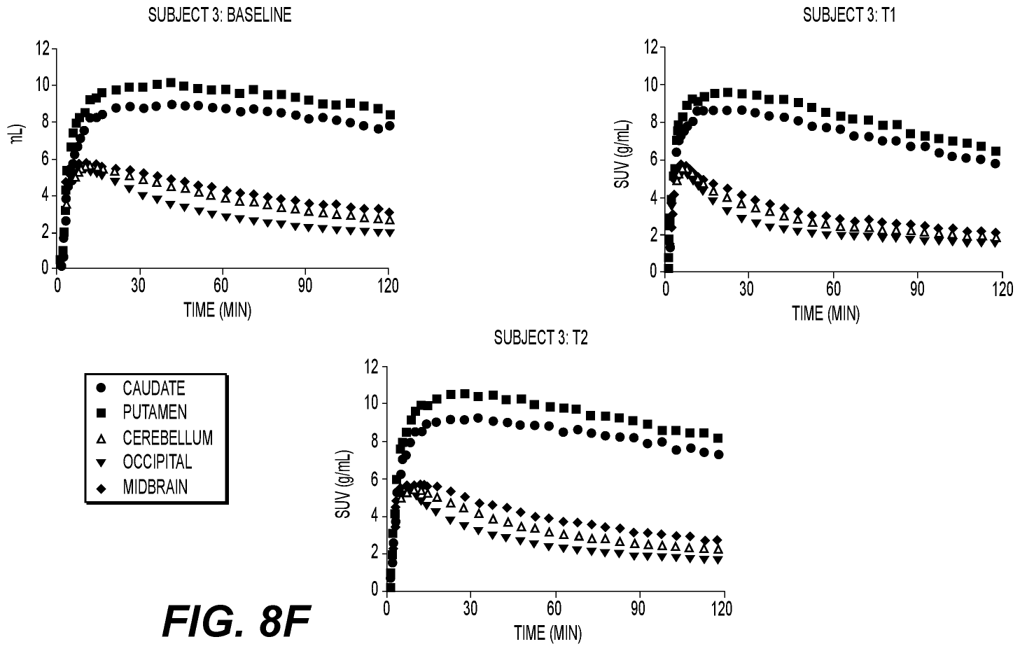

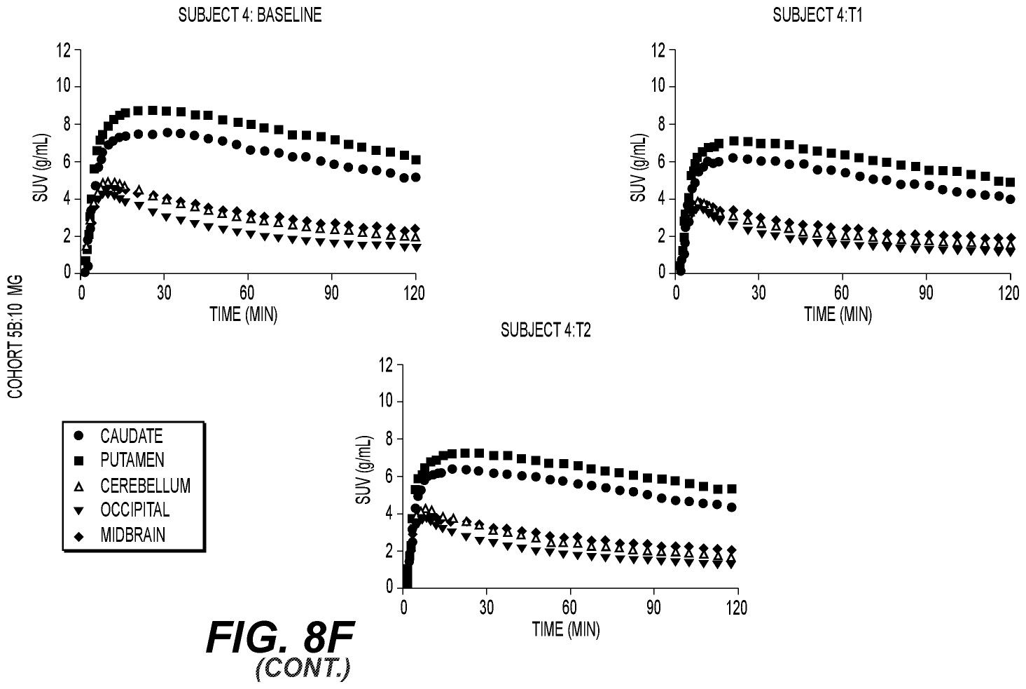

Twelve healthy male volunteers were imaged on separate days with [¹⁸F]AV-133: at baseline, and at two post NBI-750142 dose timepoints, 1.5 h (T1) and 18 h (T2). Each cohort had two to four subjects:
Cohort 1 received 100 mg;
Cohort 2 received 200 mg;
Cohort 3 received 60 mg;
Cohort 4 received 60 mg;
Cohort 5A (Subject 1 & Subject 2) received 10 mg; and
Cohort 5B (Subject 3 & Subject 4) received 10 mg.
   The dilutions for the 60 mg cohorts were different (Cohort 3=20 mg/mL in 3 mL, Cohort 4=10 mg/mL in 6 mL). For each cohort, [¹⁸F]AV-133 SUVr images (normalized to the occipital cortex), averaged between 60-120 min, are shown at baseline, and post NBI-750142 dose (T1 and T2) in FIGS. 7A-7F. [¹⁸F]AV-133 SUVr TACs are shown for the same imaging timepoints for each cohort in FIGS. 8A-8F. At baseline, [¹⁸F]AV-133 uptake was high in the caudate and putamen (target regions), where there is an abundance of VMAT2, compared with the rest of the brain. TACs show relatively lower activity in midbrain and cerebellum, with lowest uptake in the occipital cortex. At T1 compared with each subjects' [¹⁸F]AV-133 baseline in the striatum, the 100 mg and 200 mg doses markedly reduced uptake in both subjects; 60 mg in cohorts 3 and 4 showed variability between subjects, whereby the decrease in striatal uptake was less in subject 2 compared with subject 1 in both cohorts; 10 mg in cohorts 5A and 5B did not show reduction in uptake. Across all cohorts, striatal uptake at T2 appeared to return to, or slightly exceed, baseline levels.

### Quantitative Assessments

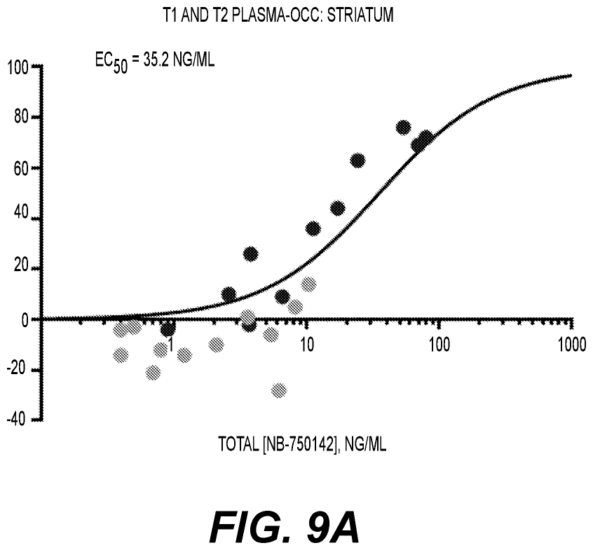

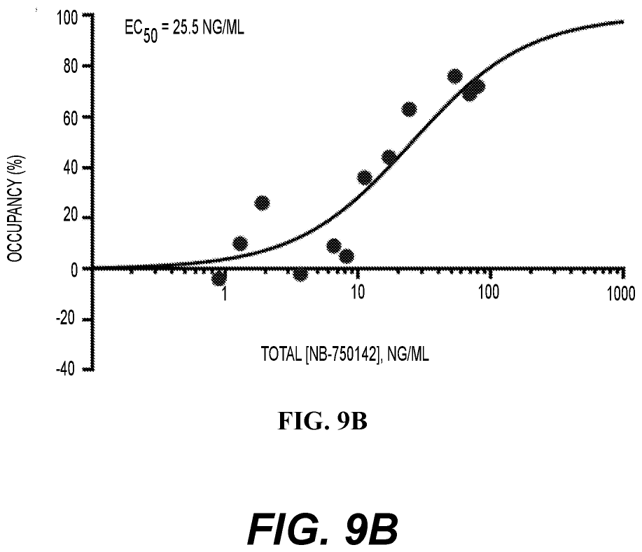

The primary outcome measure, BPND, was estimated using NI-LGA, with the occipital cortex as the reference region. BPND was estimated in the target regions, caudate and putamen, as well as a lower binding region, the midbrain. Since the cerebellum is also a known reference region for [¹⁸F]AV-133, cerebellar BPND was not assessed. In addition, the cerebellar cortex had slightly higher uptake compared with the occipital cortex, observed in the SUVr images and TACs. [¹⁸F]AV-133 BPND at baseline, T1 and T2, as well as VMAT2 occupancy by NBI-750142 at T1 and T2 are provided in Table 3-2. Occupancy at T2 in striatum for subject 1 and subject 2, respectively, were as follows:
Cohort 1 at 100 mg: 1% and 14%;
Cohort 2 at 200 mg: −28% and −6%;
Cohort 3 at 60 mg: −14% and −21%;
Cohort 4 at 60 mg: −10% and −12%;
Cohort 5A at 10 mg: −3% and −4%; and
Cohort 5B at 10 mg: −14% (subject 3) and 5% (subject 4).
   Occupancy in the midbrain mirrored the striatum, where (positive) occupancy was observed at T1; however, apparent negative occupancy was calculated at T2. The Eₘₐₓ model (see Equation 3-2) was used to describe the relationship between NBI-750142 plasma concentration and VMAT2 striatal occupancy, and EC₅₀ was estimated. FIGS. 9A-9C show NBI-750142 plasma concentration plotted against T1 and T2 occupancy, T1 occupancy only and T2 occupancy only. EC₅₀ was estimated to be 25.5 ng/mL using T1 occupancy values alone and 35.2 ng/mL using both T1 and T2 occupancy values. The Eₘₐₓ model was unable to fit the occupancy data at only T2.

TABLE 3-2
[¹⁸F]AV-133 BPND at baseline, T1 and T2, and NBI-750142 Occupancy (Occ) at T1 and T2

| | | | | | T1 | T2 | | | | T1 | T2 |
|---|---|---|---|---|---|---|---|---|---|---|---|
| Cohort | Region | Baseline | T1 | T2 | Occ | Occ | Baseline | T1 | T2 | Occ | Occ |
| | SUBJECT 1 | | | | | | SUBJECT 2 | | | | |
| 1 | Caudate | 1.19 | 0.34 | 1.15 | 72% | 3% | 1.87 | 0.38 | 1.60 | 80% | 15% |
| 100 mg | Putamen | 2.07 | 0.95 | 2.11 | 54% | −2% | 2.42 | 0.66 | 2.09 | 73% | 13% |
| | Midbrain | 0.36 | 0.15 | 0.34 | 59% | 5% | 0.51 | 0.20 | 0.36 | 60% | 30% |
| 2 | Caudate | 1.58 | 0.40 | 2.06 | 75% | −30% | 1.78 | 0.51 | 1.91 | 72% | −7% |
| 200 mg | Putamen | 2.38 | 0.74 | 3.00 | 69% | −26% | 2.68 | 0.88 | 2.81 | 67% | −5% |
| | Midbrain | 0.37 | 0.14 | 0.49 | 63% | −33% | 0.57 | 0.23 | 0.53 | 60% | 7% |
| 3 | Caudate | 1.67 | 1.04 | 1.93 | 37% | −15% | 1.72 | 1.62 | 2.07 | 6% | −20% |
| 60 mg | Putamen | 1.97 | 1.31 | 2.22 | 34% | −12% | 2.20 | 2.14 | 2.70 | 3% | −22% |
| | Midbrain | 0.37 | 0.20 | 0.39 | 47% | −5% | 0.52 | 0.44 | 0.64 | 16% | −22% |
| 4 | Caudate | 1.56 | 0.78 | 1.69 | 50% | −8% | 1.42 | 1.26 | 1.61 | 11% | −13% |
| 60 mg | Putamen | 1.80 | 1.10 | 2.01 | 39% | −12% | 1.87 | 1.73 | 2.09 | 8% | −12% |
| | Midbrain | 0.43 | 0.20 | 0.44 | 55% | −1% | 0.38 | 0.28 | 0.42 | 27% | −12% |
| 5A | Caudate | 1.55 | 1.14 | 1.58 | 27% | −2% | 2.22 | 1.98 | 2.29 | 11% | −3% |
| 10 mg | Caudate | 2.38 | 1.76 | 2.48 | 26% | −4% | 3.16 | 2.88 | 3.34 | 9% | −6% |
| | Midbrain | 0.53 | 0.31 | 0.52 | 41% | 1% | 0.54 | 0.43 | 0.65 | 20% | −19% |
| | SUBJECT 3 | | | | | | SUBJECT 4 | | | | |
| 5B | Caudate | 2.47 | 2.51 | 2.81 | −2% | −14% | 2.26 | 2.35 | 2.16 | −4% | 4% |
| 10 mg | Putamen | 2.85 | 2.90 | 3.25 | −2% | −14% | 2.93 | 3.03 | 2.75 | −3% | 6% |
| | Midbrain | 0.49 | 0.40 | 0.56 | 19% | −14% | 0.52 | 0.52 | 0.53 | 0% | −2% |

### Post Hoc Analysis: Upregulation Model

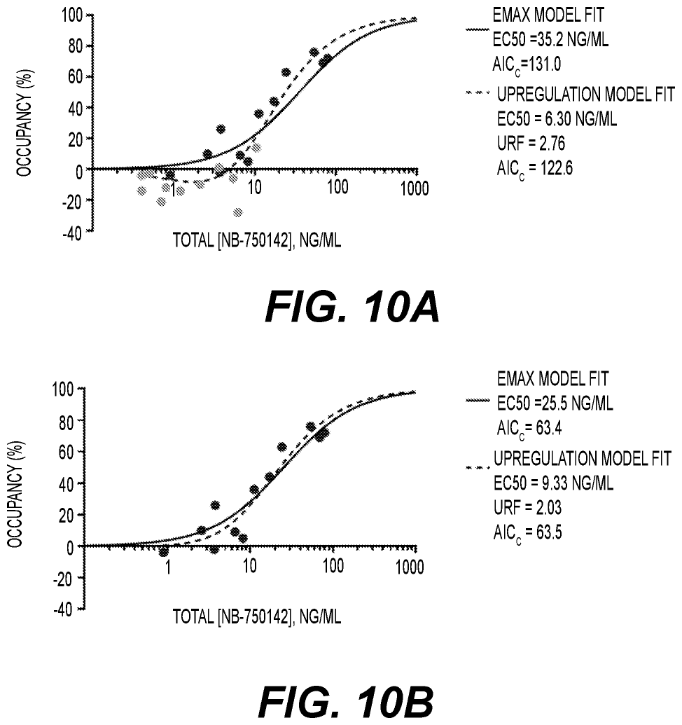

After observing apparent negative occupancy across cohorts, mainly at T2, and the inability to fit the Eₘₐₓ model to T2 data, post hoc exploratory analysis applying an Upregulation model (Equation 3-3; see also Gunn and Rabiner, Semin. Nucl. Med. 2017, 47: 89-98) was evaluated. Negative occupancies at T2 were attributed to higher striatal BPND at T2 compared with baseline. A comparison of data fits using the conventional Eₘₐₓ (Equation 3-2) and enhanced Upregulation models (Equation 3-3) is shown in FIG. 10A. The Upregulation model estimated an EC₅₀ of 6.3 ng/mL and URF of 2.76, using both T1 and T2 data. The Upregulation model better described the data using both T1 and T2 occupancy as a function of NBI-750142 plasma concentration, compared with the Eₘₐₓ model based on the visual assessment of the fits and the corrected Akaike Information Criterion (AICc) (Upregulation model: AICc=123 vs Eₘₐₓ model: AICc=131). AICc does not favor the Upregulation model over the Eₘₐₓ model when each one is fit to the T1 data alone. However, a lower EC₅₀ is estimated with the Upregulation model, as shown in FIG. 10B).

### SUMMARY AND CONCLUSIONS

In this Phase 1 open-label study, twelve healthy adult male subjects were imaged with [¹⁸F]AV-133 to investigate vesicular monoamine transporter 2 (VMAT2) target occupancy after single oral doses of NBI-750142. Occupancy in the striatum (average occupancy of caudate and putamen) ranged from −4% (10 mg) to 76% (100 mg) at T1 and from −28% (200 mg) to 14% (100 mg) at T2. The relationship between VMAT2 target occupancy and NBI-750142 plasma concentrations was assessed with the Eₘₐₓ model. EC₅₀ was estimated to be 25.5 ng/mL using T1 occupancy and 35.2 ng/mL using both T1 and T2 occupancies. However, EC₅₀ could not be estimated with T2 occupancy data alone.

Many calculated T2 occupancies were negative, even though NBI-750142 plasma concentrations remained above level of quantitation at T2, in the range of approximately 0.3-10 ng/mL. Negative occupancy observed in a PET study can result when baseline levels of binding (BPND) at the time of a ‘drug’ scan are no longer consistent with the earlier baseline scan measurements. Test-retest for BPND of [¹⁸F]AV-133 is 9.4% (see e.g., Freeby et al., Mol. Imaging Biol. 2016, April; 18(2):292-301). This suggests that the trend in negative occupancy at T2 observed in this study are different from 0%. Without being bound by theory, one possible cause for this discrepancy is an upregulation in the number of radiotracer-accessible VMAT2 binding sites following administration of drug. To address this hypothesis, post-hoc exploratory analysis using an Upregulation model was performed. The analysis using both T1 and T2 occupancies estimated an EC₅₀ of 6.3 ng/mL and upregulation factor of 2.76. Compared to the Eₘₐₓ model, the Upregulation model better described the relationship between NBI-750142 plasma concentration and T1 and T2 occupancy. This conclusion was based on visual assessment of the model fits and on the “corrected AIC” standard goodness of fit metric (Upregulation model: AICc=123 vs Emax model: AICc=131). AIC is a measure of goodness of fit that is penalized for number of parameters. Corrected AIC is preferred for small ‘n’ studies. The results suggest that the Eₘₐₓ model, even if applied only to the T1 data, may give an EC₅₀ value greater than the true EC₅₀.

In conclusion, substantial occupancy of VMAT2 by NBI-750142 was measured in the living human brains of healthy volunteers with [¹⁸F]AV-133 and PET imaging. Application of an Eₘₐₓ model to T1 data (1.5 h post NBI-750142) alone, yielded an estimated EC₅₀ of 25.5 ng/mL, while T2 data alone were not analyzable using the Eₘₐₓ model. An exploratory analysis of all the data (from T1 and T2) was also performed with a model that allows for upregulation of VMAT2 sites post-drug. The EC₅₀ from the latter analysis was 6.5 ng/mL. While exploratory, the findings of the Upregulation model suggest that the Eₘₐₓ result from T1 data alone should be treated as an upper bound.

As outlined in Example 2 (see e.g., FIG. 3A), the NBI-750142 EC₅₀ in cynomolgus monkey PET was 11 ng/mL. This value is intermediate between the EC₅₀ values obtained in humans using two different models, and within 2-3 fold of each of those values. Because of similar NBI-750142 PPB in cynomolgus monkey and human, this close relationship between monkey and human PET results also applies to EC₅₀ values using free (unbound) compound concentrations. The reasonably close match between human and cynomolgus monkey PET-derived EC₅₀s further supports the conclusions described in Examples 1-2, in which cynomolgus monkey PET EC₅₀s were applied to human PK data.

### Example 4. Valbenazine Effects on the Dopamine System in Humans, as Measured by [¹¹C]-PHNO Positron Emission Tomography (PET)

Interim imaging and tolerability data in an ongoing study were collected and analyzed in cohorts of 2-4 healthy volunteers. For each scan, participants received an injection of the D2/D3 dopamine receptor agonist radioligand [¹¹C](+)4-propyl-3,4,4a,5,6,10b-hexahydro-2H-naphtho[1,2-b][1,4]oxazin-9-ol ([¹¹C]-PHNO), followed by 90 min of data acquisition using Siemens Biograph PET/CT. For post-valbenazine scans, participants received an oral dose of valbenazine 6-8 hours before the administration of [¹¹C]-PHNO, with PET imaging occurring around the time of maximal valbenazine plasma concentration. The binding potential relative to the non-displaceable binding (BP_{ND}) in the putamen, caudate, and ventral striatum, was used as the primary endpoint. The cerebellum was used as the reference region to estimate the regional BP_{ND}. Decreases in synaptic dopamine following valbenazine administration corresponded to an increase in [¹¹C]-PHNO BP_{ND} relative to baseline (ΔBP_{ND}). Plasma concentrations of valbenazine and (+)-α-dTBZ (dihydrotetrabenazine), the active metabolite of valbenazine, were measured at the start and end of each post-valbenazine PET scan. The mean plasma (+)-α-dTBZ concentration (Cₐᵥₑ) was matched to the ΔBP_{ND} for each participant to provide an exposure-response curve.

Nine participants (5 male, 4 female) received between 40-160 mg valbenazine, which resulted in plasma (+)-α-dTBZ Cₐᵥₑ between approximately 10-60 ng/mL. Interim analysis showed eight participants displayed valbenazine-induced, dose-dependent increases in [¹¹C]-PHNO ΔBP_{ND} (21-44%). Higher exposures to (+)-α-dTBZ from higher doses of valbenazine resulted in greater ΔBP_{ND}, revealing a monotonic exposure-response curve. Adverse events in this study were consistent with the known safety and tolerability profile of valbenazine, as previously reported in tardive dyskinesia (TD) clinical trials.

Valbenazine appeared to decrease synaptic dopamine in a dose- and concentration-dependent manner, as indicated by an increase in [¹¹C]-PHNO ΔBP_{ND}. The approximately 20-40% [¹¹C]-PHNO ΔBP_{ND} increase observed in this study is similar to the [¹¹C]-PHNO ΔBP_{ND} seen previously following treatment with a tyrosine hydroxylase inhibitor to deplete dopamine (Caravaggio et al, Neuropsychopharmacology 2014; 39:2769). Thus, at pharmacological and therapeutic valbenazine doses, biologically meaningful dopamine decreases were observed in humans. These data will enable future exploration of the relationship between VMAT2 inhibition and the potential treatment of other central nervous system disorders.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are part of the present invention contemplated by this disclosure.

What is claimed is:

1. A method of preparing a pharmaceutical composition comprising a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, wherein a VMAT2 occupancy rate between 80-96% is indicative of the amount of the VMAT2 inhibitor being a therapeutically effective dosage; and
(c) admixing the therapeutically effective dosage of the VMAT2 inhibitor with a pharmaceutically acceptable carrier.

2. The method of claim 1, wherein VMAT2 occupancy is measured by one or more imaging techniques.

3. The method of claim 2, wherein one or more imaging techniques comprise administering to the subject an imaging agent capable of binding VMAT2 and subsequently imaging the subject.

4. The method of claim 3, wherein the imaging agent is a VMAT2 inhibitor.

5. The method of claim 1 or 2, wherein VMAT2 occupancy is measured by a positron emission tomography (PET) assay.

6. The method of claim 5, wherein the PET assay comprises administering to the subject a PET imaging agent capable of binding VMAT2 and subsequently imaging the subject.

7. The method of claim 5 or 6, wherein the PET assay comprises:
(a) administering to the subject a PET imaging agent capable of binding VMAT2;
(b) waiting a time sufficient for the PET imaging agent to bind VMAT2;
(c) imaging the subject one or more times;
(d) measuring the VMAT2 displacement of the PET imaging agent; and
(e) determining VMAT2 occupancy based on the measured VMAT2 displacement of the PET imaging agent.

8. The method of claim 7, wherein the VMAT2 displacement of the PET imaging agent is measured at one or more time points during the imaging.

9. The method of claim 7 or 8, further comprising imaging the subject prior to step (a) to obtain a baseline image.

10. The method of any one of claims 7 to 9, wherein the VMAT2 inhibitor is administered to the subject after step (a).

11. The method of any one of claims 7 to 9, wherein the VMAT2 inhibitor is administered to the subject after step (b).

12. The method of any one of claims 7 to 9, wherein the VMAT2 inhibitor is administered to the subject after step (b) and prior to step (c).

13. The method of any one of claims 6 to 12, wherein the PET imaging agent is a radiolabeled VMAT2 inhibitor.

14. The method of any one of claims 6 to 13, wherein the PET imaging agent is a [¹¹C]-radiolabeled VMAT2 inhibitor.

15. The method of any one of claims 6 to 13, wherein the PET imaging agent is a [¹⁸F]-radiolabeled VMAT2 inhibitor.

16. The method of any one of claims 6 to 13, wherein the PET imaging agent is a radiolabeled analog of a VMAT2 inhibitor selected from the group consisting of valbenazine, tetrabenazine, deutetrabenazine, dihydrotetrabenazine, NBI-750142, and AV-133.

17. The method of any one of claims 6 to 13, wherein the PET imaging agent is a [¹¹C]- or [¹⁸F]-radiolabeled analog of a VMAT2 inhibitor selected from the group consisting of valbenazine, tetrabenazine, deutetrabenazine, dihydrotetrabenazine, NBI-750142, and AV-133.

18. The method of claim 16 or 17, wherein the radiolabeled analog of dihydrotetrabenazine is a radiolabeled analog of (+)-α-dihydrotetrabenazine.

19. The method any one of claims 6 to 18, wherein the PET imaging agent is [¹⁸F]-AV-133.

20. The method of any one of claims 1 to 19, further comprising measuring the plasma concentration of the VMAT2 inhibitor in the subject.

21. The method of claim 20, wherein the plasma concentration of the VMAT2 inhibitor is measured at one or more time points during the imaging of step (c).

22. The method of claim 20, wherein the plasma concentration of the VMAT2 inhibitor is measured at one or more time points prior to the imaging of step (c).

23. The method of claim 20, wherein the plasma concentration of the VMAT2 inhibitor is measured at one or more time points during the imaging of step (c) and prior to the imaging of step (c).

24. The method of any one of claims 20 to 23, wherein the plasma concentration of the VMAT2 inhibitor is measured at one or more time points from about two hours prior to the imaging of step (c) until the end of the imaging.

25. The method of any one of claims 20 to 24, wherein the plasma concentration of the VMAT2 inhibitor is measured at one or more time points from about one hour prior to the imaging of step (c) until the end of the imaging.

26. The method of any one of claims 1 to 25, wherein the dose administered is identified as a therapeutically effective dosage if the VMAT2 occupancy is determined to be from at least 80% and no more than 95%.

27. The method of any one of claims 1 to 26, further comprising monitoring the subject for one or more symptoms associated with a treatment-emergent adverse event (TEAE) after administration of the VMAT2 inhibitor.

28. The method of claim 27, wherein the monitoring is performed for about 30 minutes to about 90 minutes after administration of the VMAT2 inhibitor.

29. The method of claim 27, wherein the monitoring is performed for about 30 minutes to about 60 minutes after administration of the VMAT2 inhibitor.

30. The method of any one of claims 1 to 29, further comprising identifying the subject as not exhibiting one or more symptoms associated with a TEAE after administration of the VMAT2 inhibitor.

31. The method of any one of claims 1 to 30, further comprising identifying the subject as not exhibiting one or more symptoms selected from ptosis, decreased activity, sedation, anxiety, nausea, akathisia, and salivation after administration of the VMAT2 inhibitor.

32. The method of any one of claims 1 to 31, wherein the subject has been identified as not exhibiting one or more symptoms associated with a TEAE after administration of the VMAT2 inhibitor.

33. The method of any one of claims 1 to 32, wherein the subject has been identified as not exhibiting one or more symptoms selected from ptosis, decreased activity, and salivation after administration of the VMAT2 inhibitor.

34. The method of any one of claims 1 to 33, wherein the dose administered is identified as a therapeutically effective dosage if VMAT2 occupancy is determined to be from at least 80% and no more than 95%.

35. The method of any one of claims 1 to 33, wherein the dose administered is identified as a therapeutically effective dosage if VMAT2 occupancy is determined to be from at least 85% and no more than 95%.

36. The method of claim 35, wherein the dose administered is identified as a therapeutically effective dosage if VMAT2 occupancy is determined to be from at least 85% and no more than 90%.

37. A method of identifying a therapeutically effective dosage of a VMAT2 inhibitor, the method comprising:
(a) administering an amount of a VMAT2 inhibitor to a subject;
(b) measuring in vivo VMAT2 occupancy of the VMAT2 inhibitor in the subject, and
(c) identifying a therapeutically effective dosage of a VMAT2 inhibitor when the VMAT2 occupancy rate of the amount of the VMAT2 inhibitor is between 80-96%.

38. The method of claim 37, wherein VMAT2 occupancy is measured by one or more imaging techniques.

39. The method of claim 38, wherein one or more imaging techniques comprise administering to the subject an imaging agent capable of binding VMAT2 and subsequently imaging the subject.

40. The method of claim 39, wherein the imaging agent is a VMAT2 inhibitor.

41. The method of claim 37 or 38, wherein VMAT2 occupancy is measured by a positron emission tomography (PET) assay.

42. The method of claim 41, wherein the PET assay comprises administering to the subject a PET imaging agent capable of binding VMAT2 and subsequently imaging the subject.

43. The method of claim 41 or 42, wherein the PET assay comprises:
(a) administering to the subject a PET imaging agent capable of binding VMAT2;
(b) waiting a time sufficient for the PET imaging agent to bind VMAT2;
(c) imaging the subject one or more times;
(d) measuring the VMAT2 displacement of the PET imaging agent; and
(e) determining VMAT2 occupancy based on the measured VMAT2 displacement of the PET imaging agent.

44. The method of claim 43, wherein the VMAT2 displacement of the PET imaging agent is measured at one or more time points during the imaging.

45. The method of claim 43 or 44, further comprising imaging the subject prior to step (a) to obtain a baseline image.

46. The method of any one of claims 43 to 45, wherein the VMAT2 inhibitor is administered to the subject after step (a).

47. The method of any one of claims 43 to 45, wherein the VMAT2 inhibitor is administered to the subject after step (b).

48. The method of any one of claims 43 to 45, wherein the VMAT2 inhibitor is administered to the subject after step (b) and prior to step (c).

49. The method of any one of claims 42 to 48, wherein the PET imaging agent is a radiolabeled VMAT2 inhibitor.

50. The method of any one of claims 42 to 49, wherein the PET imaging agent is a [¹¹C]-radiolabeled VMAT2 inhibitor.

51. The method of any one of claims 42 to 49, wherein the PET imaging agent is a [¹⁸F]-radiolabeled VMAT2 inhibitor.

52. The method of any one of claims 42 to 49, wherein the PET imaging agent is a radiolabeled analog of a VMAT2 inhibitor selected from the group consisting of valbenazine, tetrabenazine, deutetrabenazine, dihydrotetrabenazine, NBI-750142, and AV-133.

53. The method of any one of claims 42 to 49, wherein the PET imaging agent is a [¹¹C]- or [¹⁸F]-radiolabeled analog of a VMAT2 inhibitor selected from the group consisting of valbenazine, tetrabenazine, deutetrabenazine, dihydrotetrabenazine, NBI-750142, and AV-133.

54. The method of claim 52 or 53, wherein the radiolabeled analog of dihydrotetrabenazine is a radiolabeled analog of (+)-α-dihydrotetrabenazine.

55. The method any one of claims 42 to 54, wherein the PET imaging agent is [¹⁸F]-AV-133.

56. The method of any one of claims 37 to 55, further comprising measuring the plasma concentration of the VMAT2 inhibitor in the subject.

57. The method of claim 56, wherein the plasma concentration of the VMAT2 inhibitor is measured at one or more time points during the imaging of step (c).

58. The method of claim 56, wherein the plasma concentration of the VMAT2 inhibitor is measured at one or more time points prior to the imaging of step (c).

59. The method of claim 56, wherein the plasma concentration of the VMAT2 inhibitor is measured at one or more time points during the imaging of step (c) and prior to the imaging of step (c).

60. The method of any one of claims 56 to 59, wherein the plasma concentration of the VMAT2 inhibitor is measured at one or more time points from about two hours prior to the imaging of step (c) until the end of the imaging.

61. The method of any one of claims 56 to 60, wherein the plasma concentration of the VMAT2 inhibitor is measured at one or more time points from about one hour prior to the imaging of step (c) until the end of the imaging.

62. The method of any one of claims 37 to 61, wherein the dose administered is identified as a therapeutically effective dosage if the VMAT2 occupancy is determined to be from at least 80% and no more than 95%.

63. The method of any one of claims 37 to 62, further comprising monitoring the subject for one or more symptoms associated with a treatment-emergent adverse event (TEAE) after administration of the VMAT2 inhibitor.

64. The method of claim 63, wherein the monitoring is performed for about 30 minutes to about 90 minutes after administration of the VMAT2 inhibitor.

65. The method of claim 63, wherein the monitoring is performed for about 30 minutes to about 60 minutes after administration of the VMAT2 inhibitor.

66. The method of any one of claims 37 to 65, further comprising identifying the subject as not exhibiting one or more symptoms associated with a TEAE after administration of the VMAT2 inhibitor.

67. The method of any one of claims 37 to 66, further comprising identifying the subject as not exhibiting one or more symptoms selected from ptosis, decreased activity, sedation, anxiety, nausea, akathisia, and salivation after administration of the VMAT2 inhibitor.

68. The method of any one of claims 37 to 67, wherein the subject has been identified as not exhibiting one or more symptoms associated with a TEAE after administration of the VMAT2 inhibitor.

69. The method of any one of claims 37 to 68, wherein the subject has been identified as not exhibiting one or more symptoms selected from ptosis, decreased activity, and salivation after administration of the VMAT2 inhibitor.

70. The method of any one of claims 37 to 69, wherein the dose administered is identified as a therapeutically effective dosage if VMAT2 occupancy is determined to be from at least 80% and no more than 90%.

71. The method of claim any one of claims 37 to 69, wherein the dose administered is identified as a therapeutically effective dosage if VMAT2 occupancy is determined to be from at least 85% and no more than 95%.

72. The method of claim 71, wherein the dose administered is identified as a therapeutically effective dosage if VMAT2 occupancy is determined to be from at least 85% and no more than 90%.

73. The method of any one of claims 1-72, further comprising measuring synaptic dopamine in the subject by
(iii) administering radioligand [¹¹C](+)4-propyl-3,4,4a,5,6,10b-hexahydro-2H-naphtho[1,2-b][1,4]oxazin-9-ol ([¹¹C]-PHNO) to the subject; and
(iv) image scanning the subject;
wherein a 20-45% increase in ([¹¹C]-PHNO binding potential relative to the non-displaceable binding ([¹¹C]-PHNO BP_{ND}) corresponds to a decrease in synaptic dopamine and is indicative of the amount of the VMAT2 inhibitor being a therapeutically effective dosage.

74. A method of preparing a pharmaceutical composition comprising a therapeutically effective dosage of a VMAT2 inhibitor, the method comprising:
admixing the therapeutically effective dosage of the VMAT2 inhibitor with a pharmaceutically acceptable carrier;
wherein the therapeutically effective dosage of the VMAT2 inhibitor was identified by
measuring in vivo VMAT2 occupancy of the VMAT2 inhibitor in a subject previously administered with an amount of the VMAT2 inhibitor, wherein a VMAT2 occupancy rate between 80-96% was indicative of the amount of the VMAT2 inhibitor being a therapeutically effective dosage.

75. The method of claim 74, further comprising measuring synaptic dopamine in a subject previously administered with an amount of the VMAT2 inhibitor by
(i) administering radioligand [¹¹C](+)4-propyl-3,4,4a,5,6,10b-hexahydro-2H-naphtho[1,2-b][1,4]oxazin-9-ol ([¹¹C]-PHNO) to the subject; and
(ii) image scanning the subject;
wherein a 20-45% increase in ([¹¹C]-PHNO binding potential relative to the non-displaceable binding ([¹¹C]-PHNO BP_{ND}) corresponded to a decrease in synaptic dopamine and was indicative of the amount of the VMAT2 inhibitor being a therapeutically effective dosage.
